(19) United States
(12) Patent Application Publication
(10) Pub. No.: US 2026/0193149 A1
(43) Pub. Date: Jul. 9, 2026

(54) AGRICULTURALLY USEFUL MINERAL AMINO ACID COMPLEXES

(71) Applicant: Statera Ag, LLC, Noblesville, IN (US)

(72) Inventors: Adam Froetschner, Dodge City, KS (US); Jared Froetschner, Fishers, IN (US); Ryan Stapleton, Meade, KS (US); Jarret Stapleton, Meade, KS (US)

(21) Appl. No.: 18/868,640

(22) PCT Filed: May 19, 2023
(86) PCT No.: PCT/US2023/067219

§ 371 Date: Nov. 22, 2024

Related U.S. Application Data
(60) Provisional application No. 63/365,213, filed on May 24, 2022.
(60) Provisional application No. 63/378,344, filed on Oct. 4, 2022.
(60) Provisional application No. 63/384,524, filed on Nov. 21, 2022.

(51) Int. Cl. C05F 11/00; C05D 9/02; C05G 3/50; C05G 5/23; C07C 227/16; C07C 229/26

(52) U.S. Cl. CPC C05F 11/00; C05D 9/02; C05G 3/50; C05G 5/23; C07C 227/16; C07C 229/26

(57) ABSTRACT

Provided herein are mineral:amino acid complexes wherein the mineral comprises a macronutrient or micronutrient of agrochemical interest. The amino acid may comprise, for example, glycine or lysine. The mineral may comprise, for example, boron. The present disclosure also provides a method of making the mineral:amino acid complexes as well as a method of applying a mineral:amino acid complex to a plant, seed, or soil.

### CROSS-REFERENCE TO RELATED APPLICATIONS

The present application claims priority to U.S. Provisional Patent Application No. 63/365,213, filed May 24, 2022, to U.S. Provisional Patent Application No. 63/378,344, filed Oct. 4, 2022, and to U.S. Provisional Patent Application No. 63/384,524, filed Nov. 21, 2022, each of which is incorporated herein by reference in its entirety.

### FIELD

The present disclosure generally relates to mineral:amino acid complexes, treatment compositions comprising a mineral:amino acid complex, a method of applying the treatment composition to a plant, seed, or soil, and a method of making a mineral:amino acid complex.

### BACKGROUND

Mineral deficiencies in plants can cause undesirable phenotypic characteristics and reduced yield. That is, nutrient deficiencies can stunt growth and normal functioning and cause overall health of a plant to decline. Nutrient deficiencies in plants occur when the available nutrient levels are not sufficient to meet the plants' requirements.

Plant nutrient requirements depend largely on the target nutrient: macronutrients require larger concentrations in plant tissues and micronutrients are typically present in smaller quantities. Macronutrients generally include carbon, hydrogen, oxygen, phosphorous, potassium, nitrogen, sulfur, calcium, magnesium, and silicon. Typical micronutrients of interest include iron, molybdenum, boron, copper, manganese, sodium, zinc, nickel, chlorine, selenium, vanadium, and cobalt.

In the foliar amendment market, several chelates are readily available, including mineral:EDTA and mineral:EDDHA products. While EDTA is a coordination complex, it has a unique behavior of requiring a substitution of one element for another. In other words, if a Zn-EDTA complex is applied to a growing crop, then zinc is released if the plant provides another positive elemental species to replace the complex-bound zinc. This is undesirable.

There is therefore a need in the art for an improved method of delivering mineral nutrients to plants in order to maintain plant health and vigor.

### SUMMARY

In one aspect, provided herein is a mineral:amino acid complex that is useful, for example, for application to crop plants. In some embodiments, the amino acid complex corresponds in structure to Formula I:

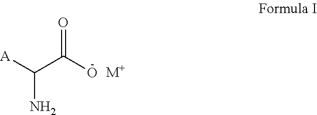

wherein A is an amino acid side chain; and M is a mineral selected from the group consisting of zinc, copper, manganese, boron, molybdenum, iron, and potassium. For example, A may be lysyl (so that the anion is lysinate). In a preferred embodiment, A is lysyl (i.e., (CH₂)₄NH₂), M is boron, and the mineral:amino acid complex may be referred to as boron lysinate.

For example, provided herein is a mineral lysinate complex of Formula Ia:

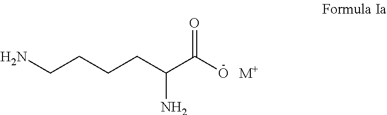

wherein M is a mineral or a cation comprising a mineral, wherein said mineral is selected from the group consisting of zinc, copper, manganese, boron, molybdenum, iron, potassium, or a combination thereof.

Also provided herein is a mineral lysinate complex of Formula IIa:

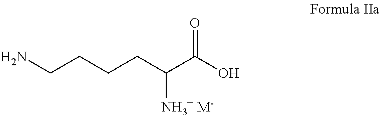

wherein M is a mineral or an anion comprising a mineral, wherein said mineral is selected from the group consisting of zinc, copper, manganese, boron, molybdenum, iron, potassium, or a combination thereof.

For example, the mineral lysinate complex of Formula IIa may have a structure corresponding to Formula IIa-i.

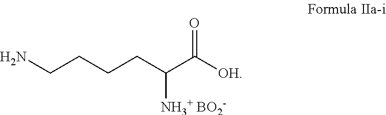

Also provided herein is a boron lysinate ligand of Formula IIb-i or Formula IIc-i.

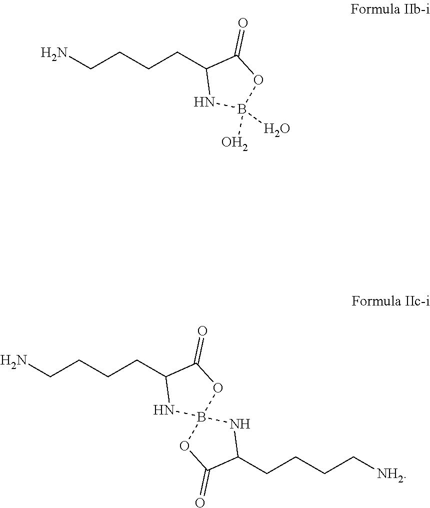

Also provided herein is an aqueous agrochemical composition suitable for application to a plant, a seed, or soil, the composition comprising a mineral lysinate complex of Formula I, Formula Ia, Formula Ia-i, Formula Ia-ii, Formula Ia-iii, Formula Ia-iv, Formula II, Formula IIa, Formula IIa-i, Formula IIb, Formula IIb-i, Formula IIc, Formula IIc-i, or a combination of any thereof in a concentration of from about 1% to about 20% by weight of the composition, and water in in in a concentration of from about 80% to about 99% by weight of the composition. The composition is preferably in the form of an aqueous solution.

Also provided herein is a method of preparing a mineral amino acid complex, the method comprising an amino acid addition step wherein an amino acid is mixed with water, thereby forming a reaction mixture, and a mineral reactant addition step wherein a mineral reactant is added to the reaction mixture, wherein said amino acid is selected from the group consisting of glycine and lysine, and wherein said mineral reactant comprises a mineral selected from the group consisting of zinc, copper, manganese, boron, molybdenum, iron, and potassium.

Also provided herein is a method of supplying a plant, a seed, or soil with an agriculturally useful mineral, the method comprising forming an application mixture comprising an amino acid complex of Formula I, Formula Ia, Formula Ia-i, Formula Ia-ii, Formula Ia-iii, Formula Ia-iv, Formula II, Formula IIa, Formula IIa-i, Formula IIb, Formula IIb-i, Formula IIc, Formula IIc-i, or a combination of any thereof and a solvent, and applying the application mixture to the plant, seed, or soil.

Also provided herein is an aqueous agrochemical composition comprising a first mineral lysinate complex of Formula Ia wherein M is iron, a second mineral lysinate complex of Formula Ia wherein M is manganese or magnesium, and water. The molar ratio of the first amino acid complex to the second amino acid complex is preferably from about 2:1 to about 1:2, and most preferably about 1:1.

Also provided herein is a method of treating iron chlorosis in a plant, the method comprising applying to a plant an effective amount of an agrochemical composition comprising (1) iron lysinate, and (2) magnesium lysinate, manganese lysinate, or a combination thereof.

The agrochemical composition is preferably applied at a rate of at least about 50 grams of the mineral lysinate complex per acre, for example, at a rate of at least about 400 grams of the mineral lysinate complex per acre.

Also provided herein is an agrochemical composition comprising a mineral:amino acid complex as described and a solvent. The agrochemical composition may generally comprise other additives and excipients that are commonly used in fertilizer compositions, as described in further detail below.

Also provided herein is a method of making a mineral:amino acid complex as described herein. For example, the method may comprise dissolving at least about 50% of a mineral reactant in a solvent and adding the amino acid to the solvent, wherein the mineral reactant comprises the mineral M therein, the mineral M being present as an ion in solution once the mineral reactant is dissolved in the solvent.

Also provided herein is a method of applying the mineral:amino acid complex to a plant, seed, or soil. For example, the method may comprise forming an application mixture comprising the amino acid complex and a solvent, and applying the application mixture to the plant, seed, or soil.

Other objects and features will be in part apparent and in part pointed out hereinafter.

### DETAILED DESCRIPTION

Provided herein are mineral:amino acid complexes that comprise (1) a mineral or a compound comprising a mineral, and (2) an amino acid. The mineral:amino acid complexes provide significant benefits relative to traditional foliar amendment compositions. For example, it has been surprisingly discovered that if the coordination complex is a mineral:amino acid species, the plant will readily absorb both the mineral and the amino acid.

This represents a significant advantage over the foliar amendment compositions known in the prior art. For example, EDTA and EDDHA complexes require the plant to sacrifice a nutrient (e.g., Zn or Ca) to release the chelated nutrient (e.g., Fe). Mineral lysinates, in contrast, are neutral in charge and the lysine does not require a substitution (unlike EDDHA or EDTA) to release the iron. Additionally, the plant cell can utilize both the lysine and the mineral nutrient. In contrast, plants do not metabolize either EDTA or EDDHA.

For example, iron is a necessary co-factor in the production of chlorophyll in plants. Additionally, chlorophyll chelates magnesium at the center of the molecule. By providing mineral nutrients (e.g., iron and/or magnsesium) in a metabolically available form (e.g., as part of a complex with lysine), the production of chlorophyll can be restarted and phytosideraphore can be produced by the plant. This then restarts the reduction of soil iron so that the plant can continue the production of chlorophyll.

### Amino Acid Complexes

Thus, provided herein is a mineral:amino acid complex (also abbreviated herein as “amino acid complex”). The amino acid can comprise, for example, lysine. The mineral typically comprises a macronutrient or micronutrient of agrochemical interest. The mineral can comprise, for example, calcium, magnesium, silicon, iron, molybdenum, boron, copper, manganese, sodium, zinc, nickel, chlorine, selenium, vanadium, cobalt, or a combination of any thereof. More specifically, the mineral can comprise, for example, zinc, copper, manganese, boron, molybdenum, iron, potassium, or a combination thereof. For example, the mineral may comprise boron. In a preferred embodiment, the mineral is boron, the amino acid is lysine, and the mineral:amino acid complex may be referred to as boron lysinate.

As used herein, the term “mineral:amino acid complex” refers to a chemical association of (1) a mineral M and (2) an amino acid or the conjugate base of an amino acid. The chemical association may be covalent or ionic in character. For example, the mineral:amino acid complex can comprise a covalently bonded molecule, a chelation complex, or a salt. While non-limiting examples of specific structures are provided herein, the present disclosure is not related to these structures alone.

The molar ratio of the mineral to the amino acid in the mineral:amino acid complex may vary. For example, the molar ratio of the mineral to the amino acid may be about 1:1. More broadly, the molar ratio of the mineral to the amino acid may vary from about 0.25:1 to about 8:1, from about 0.25:1 to about 4:1, from about 0.5:1 to about 2:1, or from about 0.75:1 to about 1.5:1.

For example, provided herein is a complex corresponding in structure to Formula I:

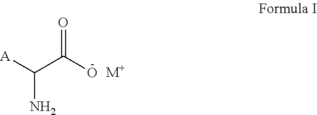

wherein A is an amino acid side chain and M is a mineral or a cation comprising a mineral, wherein said mineral is selected from the group consisting of zinc, copper, manganese, boron, molybdenum, iron, potassium, or a combination thereof. As non-limiting examples, when the amino acid is glycine, A is hydrogen; and when the amino acid is lysine, A is lysyl (i.e., (CH₂)₄NH₂).

As a non-limiting example, the amino acid complex of Formula I may correspond in structure to Formula Ia:

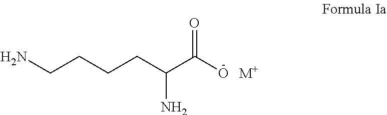

wherein M is a mineral or a cation comprising a mineral, wherein said mineral is selected from the group consisting of zinc, copper, manganese, boron, molybdenum, iron, potassium, or a combination thereof. In preferred embodiments, M is a transition metal selected from the group consisting of iron, zinc, manganese, and molybdenum.

For example, in Formula Ia, M may be iron as represented by Formula Ia-i.

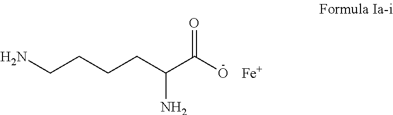

As a further example, in Formula Ia, M may be zinc as represented by Formula Ia-ii.

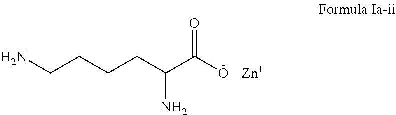

As a further example, in Formula Ia, M may be manganese as represented by Formula Ia-iii.

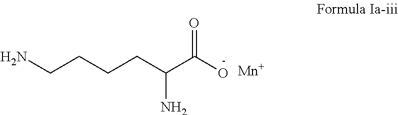

As a further example, in Formula Ia, M may be molybdenum as represented by Formula Ia-iv.

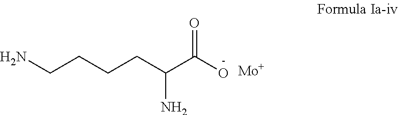

Also provided herein is a complex corresponding in structure to Formula IL:

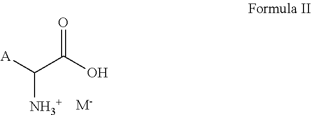

wherein A is an amino acid side chain and M is a mineral or an anion comprising a mineral, wherein said mineral is selected from the group consisting of zinc, copper, manganese, boron, molybdenum, iron, potassium, or a combination thereof. As non-limiting examples, when the amino acid is glycine, A is hydrogen; and when the amino acid is lysine, A is lysyl (i.e., (CH₂)₄NH₂).

For example, A can be lysyl, as represented in Formula IIa below.

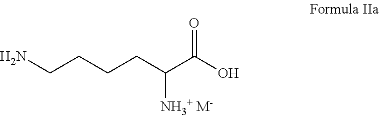

As a specific example, in Formula IIa, M can be a metaborate anion, as represented in Formula IIa-i below.

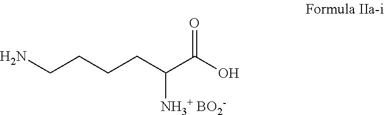

In addition to the exemplary structures disclosed above as Formula I, Formula Ia, Formula Ia-i, Formula Ia-ii, Formula Ia-iii, Formula Ia-iv, Formula II, and Formula II-i, the amino acid may interact with the mineral in a variety of other different ways. For example, the amino acid may interact with water molecules and the amino acid. The mineral may also form a bidentate ligand with one or more of the amino acid molecules.

For example, in Formula IIa, the mineral M may form two monodentate ligands with water and one bidentate ligand with lysine, as represented by Formula IIb.

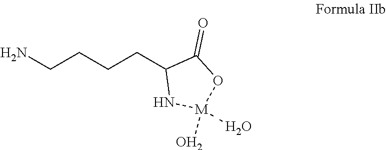

As a particular example, in Formula IIb, when M is boron, boron may form two monodentate ligands with water and one bidentate ligand with lysine, represented by Formula IIb-i.

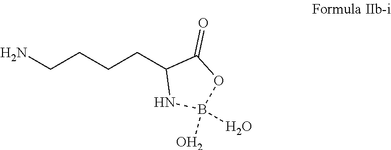

Alternatively, or in addition to Formula IIb, the mineral M may form two bidentate ligands with lysine, represented by Formula IIc.

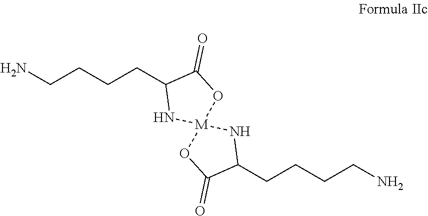

As a particular example, in Formula IIc, when M is boron, boron may form two bidentate ligands with lysine, represented by Formula IIc-i.

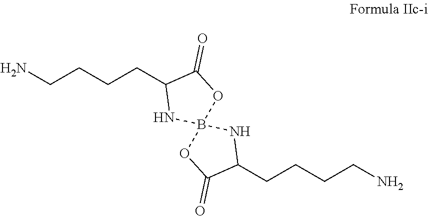

### Agrochemical Compositions

As described, provided herein is an agrochemical composition comprising an effective amount of a mineral:amino acid complex. As non-limiting examples, the mineral:amino acid complex may correspond in structure to Formula I, Formula Ia, Formula Ta-i, Formula Ia-ii, Formula Ia-iii, Formula Ia-iv, Formula II, Formula IIa, Formula IIa-i, Formula IIb, Formula IIb-i, Formula IIc, Formula IIc-i, or a combination of any thereof.

The agrochemical composition is preferably in the form of an aqueous solution comprising the mineral:amino acid complex dissolved in water.

Alternatively, the agrochemical composition may be in the form of an emulsion, a suspension, a granular composition, or a powder. The agrochemical composition may comprise a solvent, for example, water, ethanol, methanol, acetone, toluene, and the like. Preferably, the solvent comprises water.

The agrochemical compositions can be formulated as concentrate compositions or ready-to-use compositions. By “ready-to-use,” it is meant that the composition is provided in a form that requires no additional dilution by the user, and is ready for application.

### Ready-to-Use Formulations

The ready-to-use formulation is preferably an aqueous composition. More particularly, the ready-to-use formulation is preferably in the form of an aqueous solution comprising the mineral:amino acid complex dissolved in water.

For example, in a ready-to-use formulation, the concentration of the mineral:amino acid complex may be at least about 0.1 percent by weight, at least about 0.5 percent by weight, at least about 1 percent by weight, at least about 2 percent by weight, at least about 5 percent by weight, or at least about 10 percent by weight.

In a ready-to-use formulation, the concentration of the mineral:amino acid complex may be no greater than about 40 percent by weight, no greater than about 30 percent by weight, no greater than about 25 percent by weight, no greater than about 20 percent by weight, no greater than about 15 percent by weight, no greater than about 10 percent by weight, no greater than about 8 percent by weight, no greater than about 6 percent by weight, or no greater than about 5 percent by weight.

As non-limiting examples, the concentration of the mineral:amino acid complex may range from about 0.5 percent by weight to about 20 percent by weight, from about 0.5 percent by weight to about 10 percent by weight, from about 0.5 percent by weight to about 5 percent by weight, or from about 1 percent by weight to about 5 percent by weight of the ready-to-use formulation.

### Liquid Concentrate Formulations

The liquid concentrate formulation is preferably an aqueous composition. More particularly, the concentrate formulation is preferably in the form of an aqueous solution comprising the mineral:amino acid complex dissolved in water.

In a liquid concentrate formulation, the concentration of the mineral:amino acid complex is typically be at least about 10 percent by weight. For example, the concentration of the mineral:amino acid complex may be at least about 15 percent by weight, at least about 20 percent by weight, at least about 25 percent by weight, at least about 30 percent by weight, or at least about 35 percent by weight.

In a liquid concentrate formulation, the concentration of the mineral:amino acid complex typically does not exceed the lesser of about 70 percent by weight of the formulation, or the solubility limit of the mineral:amino acid complex in the selected solvent (e.g., water).

### Adjuvants, Excipients, and Other Optional Components

Generally, the amino acid complex compositions described herein can comprise any adjuvants, excipients, or other desirable components known in the art. As an example, the compositions described herein can further comprise a wetting agent, an antifoaming agent, a buffering agent, a fixing agent, a preservative, an antioxidant, a surfactant, or a combination thereof. When present, the additional component(s) can be provided in a total concentration of from about 0.1 percent to about 50 percent by weight of the composition, for example, from about 0.1 percent to about 20 percent by weight of the composition, from about 1 percent to about 20 percent by weight of the composition, or from about 1 percent to about 10 percent by weight of the composition.

Suitable wetting agents include all compounds that promote wetting and are typically used in agrochemical compositions, including, for example, an alkylnaphthalene-sulfonate, such as diisopropylnaphthalene-sulfonate and diisobutylnaphthalene-sulfonate.

Suitable antifoaming agents include all agrochemically effective foam-inhibiting compounds, such as a silicone antifoaming agent, magnesium stearate, a silicone emulsion, a long-chain alcohol, a fatty acid and its salt, and an organofluorine compound, or any mixture of any thereof.

Suitable buffering agents include all buffering agents typically used in agricultural compositions, such as, for example, monopotassium phosphate, acrylic acid, glutaric acid, gluconic acid, glycolic acid, lactic acid, carboxylated alcohol ethoxylate, an ethoxylated alkylphenol carboxylate ester, a tristyrylphenol alkoxylate carboxylate ester, a tristyrylphenol alkoxylate phosphate ester, a fatty acid, or a mixture of any thereof.

Suitable fixing agents can be based on a polyvinyl alkyl ether, for example polyvinyl methyl ether or ketones, such as benzophenone or ethylene benzophenone.

Suitable preservatives include all preservatives typically used in agricultural compositions, such as, for example, a preservative made from dichlorophen and benzyl alcohol hemiformal. Other suitable preservatives include 1,2-benzoisothiazolin-3-one, 1,2-benzispthiazolin-3-one, 5-chloro-2-methyl-4-isothiazolin-3-one, 2-methyl-4-isothiazolin-3-one, or a mixture of any thereof.

Suitable antioxidants include all antioxidants typically used in agricultural compositions, such as, for example, butylated hydroxytoluene (BHT), propyl gallate, octyl gallate, dodecyl gallate, butylated hydroxyanisole, propylparaben, sodium benzoate, 4,4′-(2,3-dimethyltetramethylene) dibrenzcatechin (nordihydroguaiaretic acid).

Suitable surfactants include all surfactants typically used in agricultural compositions and may be nonionic, anionic, cationic, or zwitterionic.

Nonionic surfactants include polyethylene oxide-polypropylene oxide block copolymers, polyethylene-polypropylene glycol, alkylpolyoxyethylene, polyethylene glycol ethers of linear alcohols, reaction products of fatty acids with ethylene oxide and/or propylene oxide, polyvinyl alcohol, polyvinylpyrrolidone, copolymers of polyvinyl alcohol and polyvinylpyrrolidone, copolymers of (meth)acrylic acid and (meth)acrylic esters, alkyl ethoxylates, alkylaryl ethoxylates, which may be optionally phosphated or neutralized with a base, poyloxyamine derivatives, nonylphenol ethoxylates, and any mixture of any thereof.

Anionic surfactants include, for example, alkali metal and alkaline earth metal salts of alkylsulfonic acid and alkylarylsulfonic acid, salts of polystyrenesulfonic acid, salts of polyvinyl sulfonic acids, salts of naphthalene sulfonic acid, formaldehyde condensates, salts of condensates of naphthalenesulfonic acid, phenolsulfonic acid and formaldehyde, salts of ligninsulfonic acid, and a mixture any thereof.

The surfactant can comprise an alkyl carboxylate, sodium stearate, sodium lauryl sarcosinate, perfluorononanoate, perfluorooctanoate, ammonium lauryl sulfate, sodium lauryl sulfate, sodium laureth sulfate, sodium myreth sulfate, docusate, perfluorooctanesulfonate, perfluorobutanesulfonate, an alkyl-aryl ether phosphate, an alkyl ether phosphate, octenidine dihydrochloride, cetrimonium bromide, cetylpyridinium chloride, benzalkonium chloride, benzethonium chloride, dimethyldioctadecylammonium chloride, dioctadecyldimethylammonium bromide, 3-[(3-cholamidopropyl)dimethylammonio]-1-propanesulfonate, cocamidopropyl hydroxysultaine, cocamidopropyl betaine, phosphtidylserine, phosphatidylethanolamine, phosphatidylcholine, a shingomyelin, a fatty alcohol, cetyl alcohol, stearyl alcohol, cetostearyl alcohol, oleyl alcohol, a polyoxyethylene glycol alkyl ether, ocatethylene glycol monodecyl ether, pentaethylene glycol monodecyl ether, a polyoxypropylene glycol alkyl ether, a glucoside alkyl ether, decyl glucoside, lauryl glucoside, octyl glucoside, polyoxyethylene gylcol octylphenol ether, an alkylene glycol, such as ethylene glycol, propylene glycol, polyethylene glycol, alkyl and alkyl lauryl polyoxyethylene glycol, an alkyl polysaccharide, an alkyl polyglucoside ester, polyethylene-polyproplyene glycol, polyoxyethylene-polyoxypropylene and polyethylene glycol, hexylene glycol, and polyoxyethylene glycol alkylphenol ether, nonoxynol-9, a glycerol alkyl ester, glyceryl laurate, a polyoxyethylene glycol sorbitan alkyl ester, polysorbate, a sorbitan alkyl ester, cocamide monoethanolamine, cocamide diethanolamine, dodecyldimethylamine oxide, a block copolymer of polyethylene glycol, a block copolymer of polypropylene glycol, poloxamer, polyethoxylated tallow amine, a polyoxyalkylene or derivative thereof, such as alkyl polyoxyethylene, methoxypolyoxyethylene, octyl polyoxyethylene, nonyl polyoxyethylene, decyl polyoxyethylene, undecyl polyoxyethylene, lauryl polyoxyethylene, tridecyl polyoxyethylene, tetradecyl polyoxyethylene, pentadecyl polyoxyethylene, hexadecyl polyoxyethylene, heptadecyl polyoxyethylene, octadecyl polyoxyethylene, coco polyoxyethylene, tallow polyoxyethylene, alkyl polyethoxylate ether, alkyl phenol ethoxylate, and a polyoxyethylene-polyoxypropylene block copolymer, an organosilicone, an alcohol ethoxylate, an alkyl aryl ethoxylate, a sulfosuccinic acid-based surfactant, or a combination of any thereof.

The composition can also comprise an additional active ingredient, such as a pesticide, a fertilizer, a plant growth regulator, a bio-control agent, a bio-stimulant, seaweed extract or a derivative thereof, or a combination of any thereof. When present, the additional active ingredient can comprise from about 1 percent to about 70 percent by weight of the composition, for example, from about 5 percent to about 70 percent by weight, from about 20 percent to about 60 percent by weight, from about 1 percent to about 50 percent by weight, from about 1 percent to about 25 percent by weight, or from about 1 percent to about 10 percent by weight of the composition.

The pesticide can comprise a fungicide, an insecticide, an acaricide, an herbicide, a nematicide, a bactericide, or a combination of any thereof.

When included in the composition, the herbicide can comprise 2,4-D, 2,4-DB, acetochlor, acifluorfen, alachlor, ametryn, atrazine, aminopyralid, benefin, bensulfuron, bensulfuron methyl, bensulide, bentazon, bispyribac sodium, bromacil, bromoxynil, butylate, carfentrazone, 2-chlorophenoxy acetic acid, chlorimuron, chlorimuron ethyl, chlorsulfuron, clethodim, clomazone, clopyralid, clopyralid acid, cloransulam, CMPP-P-DMA, cycloate, DCPA, desmedipham, dicamba, dichlobenil, diclofop, dichlorprop, dichlorprop-P, dichlorophenoxyacetic acid, 2,4-dichlorophenol, diclosulam, diflufenzopyr, dimethenamid, dimethyl amine salt of 2,4-dichlorophenoxyacetic acid, 2,4-dichlorophenoxyacetic acid ester, derivatives of 2,4-dichlorophenoxyacetic acid, diquat, diuron, DSMA, endothall, EPTC, ethalfluralin, ethofumesate, fenoxaprop, fluazifop-P, flucarbazone, flufenacet, flumetsulam, flumiclorac, flumioxazin, fluometuron, fluroxypyr, fluroxypyr 1-methyleptylester, fomesafen, fomesafen sodium salt, foramsulfuron, glufosinate, glufosinate-ammonium, glyphosate, halosulfuron, halosulfuron-methyl, hexazinone, 2-hydroxyphenoxy acetic acid, 4-hydroxyphenoxy acetic acid, imazamethabenz, imazamox, imazapic, imazaquin, imazapyr, imazethapyr, isoxaben, isoxaflutole, lactofen, linuron, MCPA, MCPB, mecoprop, mecoprop-P, mesotrione, metolachlor-s, metribuzin, metsulfuron, metsulfuron-methyl, molinate, MSMA, napropamide, naptalam, nicosulfuron, norflurazon, oryzalin, oxadiazon, oxyfluorfen, paraquat, pelargonic acid, pendimethalin, phenmedipham, picloram, primisulfuron, prodiamine, prometryn, pronamide, propanil, prosulfuron, pyrazon, pyroxasulfone, pyrithiobac, quinclorac, quizalofop, rimsulfuron, sethoxydim, siduron, simazine, sulfentrazone, sulfometuron, tribernuron, tribernuron-methyl, sulfosulfuron, tebuthiuron, terbacil, thiazopyr, thifensulfuron, thifensulfuron-methyl, thiobencarb, tralkoxydim, triallate, triasulfuron, tribenuron, triclopyr, trifluralin, triflusulfuron, or any combination thereof.

When included in the composition, the fungicide can comprise aldimorph, ampropylfos, ampropylfos potassium, andoprim, anilazine, azaconazole, azoxystrobin, benalaxyl, benodanil, benomyl, benzamacril, benzamacryl-isobutyl, benzovindflupyr, bialaphos, binapacryl, biphenyl, bitertanol, blasticidin-S, boscalid, bromuconazole, bupirimate, buthiobate, calcium polysulphide, capsimycin, captafol, captan, carbendazim, carvon, quinomethionate, chlobenthiazone, chlorfenazole, chloroneb, chloropicrin, chlorothalonil, chlozolinate, clozylacon, cufraneb, cymoxanil, cyproconazole, cyprodinil, cyprofuram, debacarb, dichlorophen, diclobutrazole, diclofluanid, diclomezine, dicloran, diethofencarb, dimethirimol, dimethomorph, dimoxystrobin, diniconazole, diniconazole-M, dinocap, diphenylamine, dipyrithione, ditalimfos, dithianon, dodemorph, dodine, drazoxolon, edifenphos, epoxiconazole, etaconazole, ethirimol, etridiazole, famoxadon, fenapanil, fenarimol, fenbuconazole, fenfuram, fenitropan, fenpiclonil, fenpropidin, fenpropimorph, fentin acetate, fentin hydroxide, ferbam, ferimzone, fluazinam, fludioxonil, flumetover, fluoromide, fluquinconazole, flurprimidol, flusilazole, flusulfamide, fluoxastrobin, flutolanil, flutriafol, folpet, fosetyl-aluminium, fosetyl-sodium, fthalide, fuberidazole, furalaxyl, furametpyr, furcarbonil, furconazole, furconazole-cis, furmecyclox, guazatine, hexachlorobenzene, hexaconazole, hymexazole, imazalil, imibenconazole, iminoctadine, iminoctadine albesilate, iminoctadine triacetate, iodocarb, iprobenfos (IBP), iprodione, irumamycin, isoprothiolane, isovaledione, kasugamycin, kresoxim-methyl, copper preparations, such as: copper hydroxide, copper naphthenate, copper oxychloride, copper sulphate, copper oxide, oxine-copper and Bordeaux mixture, mancopper, mancozeb, maneb, meferimzone, mepanipyrim, mepronil, metconazole, methasulfocarb, methfuroxam, metalzxyl, metiram, metomeclam, metsulfovax, mildiomycin, myclobutanil, myclozolin, nickel dimethyldithiocarbamate, nitrothal-isopropyl, nuarimol, ofurace, oxadixyl, oxamocarb, oxolinic acid, oxycarboxim, oxyfenthiin, paclobutrazole, pefurazoate, penconazole, pencycuron, phosdiphen, picoxystrobin, pimaricin, piperalin, polyoxin, polyoxorim, probenazole, prochloraz, procymidone, propamocarb, propanosine-sodium, propiconazole, propineb, prothiocinazole, pyraclostrobin, pyrazophos, pyrifenox, pyrimethanil, pyroquilon, pyroxyfur, quinconazole, quintozene (PCNB), a strobilurin, sulphur and sulphur preparations, tebuconazole, tecloftalam, tecnazene, tetcyclasis, tetraconazole, thiabendazole, thicyofen, thifluzamide, thiophanate-methyl, tioxymid, tolclofos-methyl, tolylfluanid, triadimefon, triadimenol, triazbutil, a triazole, triazoxide, trichlamide, triclopyr, tricyclazole, tridemorph, trifloxystrobin, triflumizole, triforine, uniconazole, validamycin A, vinclozolin, viniconazole, zarilamide, zineb, ziram and also Dagger G, OK-8705, OK-8801, a-(1,1-dimethylethyl)-(3-(2-phenoxyethyl)-1H-1,2,4-triazole-1-ethanol, a-(2,4-dichlorophenyl)-[3-fluoro-3-propyl-1H-1,2,4-triazole-1-ethanol, a-(2,4-dichlorophenyl)-[3-methoxy-a-methyl-1H-1,2,4-triazole-1-ethanol, a-(5-methyl-1,3-dioxan-5-yl)-[3-[[4-(trifluoromethyl)-phenyl]-methylene]-1H-1,2,4-triazole-1-ethanol, (5RS,6RS)-6-hydroxy-2,2,7,7-tetramethyl-5-(1H-1,2,4-triazol-1-yl)-3-octanone, (E)-a-(methoxyimino)-N-methyl-2-phenoxy-phenylacetamide, 1-isopropyl {2-methyl-1-[[[1-(4-methylphenyl)-ethyl]-amino]-carbonyl]-propyl}carbamate, 1-(2,4-dichlorophenyl)-2-(1H-1,2,4-triazol-1-yl)-ethanone-O-(phenyl methyl)-oxime, 1-(2-methyl-1-naphthalenyl)-1H-pyrrole-2,5-dione, 1-(3,5-dichlorophenyl)-3-(2-propenyl)-2,5-pyrrolidindione, 1-[(diiodomethyl)-sulphonyl]-4-methyl-benzene, 1-[[2-(2,4-dichlorophenyl)-1,3-dioxolan-2-yl]-methyl]-1H-imidazole, 1-[[2-(4-chlorophenyl)-3-phenyloxiranyl]-methyl]-1H-1,2,4-triazole, 1-[1-[2-[(2,4-dichlorophenyl)-methoxy]-phenyl]-ethenyl]-1H-imidazole, 1-methyl-5-nonyl-2-(phenylmethyl)-3-pyrrolidinole, 2′,6′-dibromo-2-methyl-4′-trifluoromethoxy-4′-trifluoro-methyl-1,3-thiazole-carboxanilide, 2,2-dichloro-N-[1-(4-chlorophenyl)-ethyl]-1-ethyl-3-methyl-cyclopropanecarboxamide, 2,6-dichloro-5-(methylthio)-4-pyrimidinyl-thiocyanate, 2,6-dichloro-N-(4-trifluoromethylbenzyl)-benzamide, 2,6-dichloro-N-[[4-(trifluoromethyl)-phenyl]-methyl]-benzamide, 2-(2,3,3-triiodo-2-propenyl)-2H-tetrazole, 2-[(1-methylethyl)-sulphonyl]-5-(trichloromethyl)-1,3,4-thiadiazole, 2-[[6-deoxy-4-O-(4-O-methyl-(3-D-glycopyranosyl)-a-D-glucopyranos yl]-amino]-4-methoxy-1H-pyrrolo[2,3-d]pyrimidine-5-carbonitrile, 2-aminobutane, 2-bromo-2-(bromomethyl)-pentanedinitrile, 2-chloro-N-(2,3-dihydro-1,1,3-trimethyl-1H-inden-4-yl)-3-pyridinecarboxamide, 2-chloro-N-(2,6-dimethylphenyl)-N-(isothiocyanatomethyl)-acetamide, 2-phenylphenol (OPP), 3,4-dichloro-i-[4-(difluoromethoxy)-phenyl]-pyrrole-2,5-dione, 3,5-dichloro-N-[cyano[(1-methyl-2-propynyl)-oxy]-methyl]-benzamide, 3-(1,1-dimethylpropyl-1-oxo-1H-indene-2-carbonitrile, 3-[2-(4-chlorophenyl)-5-ethoxy-3-isoxazolidinyl]-pyridine, 4-chloro-2-cyano-N,N-dimethyl-5-(4-methylphenyl)-1H-imidazole-1-sulphonamide, 4-methyl-tetrazolo[1,5-a]quinazolin-5(4H)-one, 8-(1,1-dimethylethyl)-N-ethyl-N-propyl-1,4-dioxaspiro[4,5]decane-2-methanamine, 8-hydroxyquinoline sulphate, 9H-xanthene-2-[(phenylamino)-carbonyl]-9-carboxylic hydrazide, bis-(1-methylethyl)-3-methyl-4-[(3-methylbenzoyl)-oxy]-2,5-thiophenedicarboxylate, cis-1-(4-chlorophenyl)-2-(1H-1,2,4-triazol-1-yl)-cycloheptanol, cis-4-[3-[4-(1,1-dimethylpropyl)-phenyl-2-methylpropyl]-2,6-dimethyl-morpholine hydrochloride, ethyl[(4-chlorophenyl)-azo]-cyanoacetate, potassium bicarbonate, methanetetrathiol-sodium salt, methyl 1-(2,3-dihydro-2,2-dimethyl-inden-1-yl)-1H-imidazole-5-carboxylate, methyl N-(2,6-dimethylphenyl)-N-(5-isoxazolylcarbonyl)-DL-alaninate, methyl N-(chloroacetyl)-N-(2,6-dimethylphenyl)-DL-alaninate, N-(2,3-dichloro-4-hydroxyphenyl)-1-methyl-cyclohexanecarboxamide, N-(2,6-dimethyl phenyl)-2-methoxy-N-(tetra hydro-2-oxo-3-furanyl)-acetamide, N-(2,6-dimethyl phenyl)-2-methoxy-N-(tetrahydro-2-oxo-3-thienyl)-acetamide, N-(2-chloro-4-nitrophenyl)-4-methyl-3-nitro-benzenesulphonamide, N-(4-cyclohexylphenyl)-1,4,5,6-tetrahydro-2-pyrimidinamine, N-(4-hexylphenyl)-1,4,5,6-tetrahydro-2-pyrimidinamine, N-(5-chloro-2-methylphenyl)-2-methoxy-N-(2-oxo-3-oxazolidinyl)-acetamide, N-(6-methoxy)-3-pyridinyl)-cyclopropanecarboxamide, N-[2,2,2-trichloro-1-[(chloroacetyl)-amino]-ethyl]-benzamide, N-[3-chloro-4,5-bis(2-propinyloxy)-phenyl]-N′-methoxy-methanimidamide, N-formyl-N-hydroxy-DL-alanine-sodium salt, 0,0-diethyl[2-(dipropylamino)-2-oxoethyl]-ethylphosphoramidothioate, O-methyl S-phenyl phenylpropylphosphoramidothioate, S-methyl 1,2,3-benzothiadiazole-7-carbothioate, and spiro[2H]-1-benzopyrane-2,1′(3′H)-isobenzofuran]-3′-one, N-trichloromethyl)thio-4-cyclohexane-1,2-dicarboximide, tetramethylthioperoxydicarbonic diamide, methyl N-(2,6-dimethylphenyl)-N-(methoxyacetyl)-DL-alaninate, 4-(2,2-difluoro-1,3-benzodioxol-4-yl)-1-H-pyrrol-3-carbonitril, or any combination thereof.

Examples of active ingredients include organic phosphorous agents, carbonate agents, carboxylates, chlorinated hydrocarbons, and materials produced from microorganisms. The additional active ingredient may comprise alanycarb, aldicarb, aldoxycarb, allyxycarb, aminocarb, bendiocarb, benfuracarb, bufencarb, butacarb, butocarboxim, butoxycarboxim, carbaryl, carbofuran, carbosulfan, cloethocarb, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, trimethacarb, XMC, xylylcarb, and triazamate; acephate, azamethiphos, azinphos (-methyl, -ethyl), aromophos-ethyl, aromfenvinfos (-methyl), autathiofos, cadusafos, carbophenothion, chlorethoxyfos, chlorfenvinphos, chlormephos, chlorpyrifos (-methyl, -ethyl), coumaphos, cyanofenphos, cyanophos, chlorfenvinphos, demeton-S-methyl, demeton-S-methyl sulphone, dialifos, diazinone, dichlofenthione, dichlorvos/DDVP, dicrotophos, dimethoate, dimethylvinphos, dioxabenzofos, disulfoton, EPN, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidone, phosphocarb, Phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion; acrinathrin, allethrin (d-cis-trans, d-trans), beta-cyfluthrin, bifenthrin, bioallethrin, bioallethrin-S-cyclopentyl-isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta), cyphenothrin, deltamethrin, empenthrin (R-isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R-trans isomer), prallethrin, profluthrin, protrifenbute, pyresmethrin, resmethrin, RU 15525, silafluofen, tau-fluvalinate, tefluthrin, terallethrin, tetramethrin (-1R-isomer), tralomethrin, transfluthrin, ZXI 8901, pyrethrins (pyrethrum); DDT; acetamiprid, clothianidin, dinotefuran, imidacloprid, nitenpyram, nithiazine, thiacloprid, thiamethoxam, nicotine, bensultap, cartap; spinosad; camphechlor, chlordane, endosulfan, gamma-HCH, HCH, heptachlor, lindane, methoxychlor; Fiproles, such as for example acetoprole, ethiprole, fipronil, pyrafluprole, pyriprole, and vaniliprole; avermectin, emamectin, emamectin benzoate, ivermectin, milbemycin, latidectin, lepimectin, selamectin, doramectin, eprinomectin, and moxidectin; diofenolan, epofenonane, fenoxycarb, hydroprene, kinoprene, methoprene, pyriproxifen, and triprene; depsipeptides, such as emodepside; chromafenozide, halofenozide, methoxyfenozide, tebufenozide; bistrifluron, chlofluazuron, diflubenzuron, fluazuron, flucycloxuron, flufenoxuron, hexaflumuron, lufenuron, novaluron, noviflumuron, penfluron, teflubenzuron, triflumuron; buprofezin; cyromazine; diafenthiuron; cyhexatin, fenbutatin-oxide; chlorfenapyr; dinitrophenols, such as for example binapacyrl, dinobuton, dinocap, DNOC; fenazaquin, fenpyroximate, pyrimidifen, pyridaben, tebufenpyrad, tolfenpyrad; hydramethylnon; dicofol; rotenones; acequinocyl, fluacrypyrim; Bacillus thuringiensis strains; tetronic acids, such as spirodiclofen, spiromesifen; tetramic acids, such as spirotetramat, 3-(2,5-dimethylphenyl)-8-methoxy-2-oxo-1-azaspiro[4.5]dec-3-en-4-yl ethyl carbonate; carboxamaides, such as flonicamid; amitraz; flubendiamide; thiocyclam hydrogen oxalate, and thiosultap-sodium.

Suitable bactericides include kasugamycin, tetracycline, oxytetracyline, streptomycin, bacterial control agents, copper fungicides, neem oil, vinegar, or a combintaion of any thereof.

For example, the fungicide can comprise a strobilurin, a conazole, or a combination thereof. The fungicide can comprise pycraclostrobin, metconazole, or a combination thereof. As another example, the fungicide can comprise azoxystrobin. Further, the fungicide can comprise trifloxystrobin, prothioconazole, or a combination thereof.

When a fertilizer is included in the compositions of the present invention, the fertilizer can comprise a foliar nitrogen fertilizer, a foliar phosphorous fertilizer, a foliar manganese fertilizer, or a combination of any thereof.

### Methods of Making

Also provided herein are methods of making an agriculturally useful composition comprising the mineral:amino acid complex as described herein. The methods generally comprise mixing a reactant containing the target mineral (“mineral reactant”) and an amino acid with a solvent (e.g., water), thereby forming a reaction mixture. The reaction mixture then undergoes a reaction step, optionally in the presence of heat, to form a composition comprising the mineral:amino acid complex.

### Solvent

The solvent should be selected such that the mineral reactant is sufficiently soluble therein. For example, the solvent is preferably selected such that the solubility limit of the mineral reactant is at least about 25 g/L, at least about 50 g/L, at least about 75 g/L, at least about 100 g/L, at least about 150 g/L, or at least about 200 g/L or greater.

As described above, the composition is preferably an aqueous composition (e.g., an aqueous solution) wherein the solvent is water.

### Mineral Reactant

The mineral reactant may comprise the mineral in its elemental form. More typically, however, the mineral reactant is a compound comprising the mineral as a constituent thereof.

The mineral reactant may comprise a salt comprising the mineral and a counterion (a “mineral salt”). For example, the mineral salt may comprise the mineral as a cation, and an anionic counterion selected from the group consisting of sulfate, sulfite, nitrate, nitrite, a halogen ion, phosphate, hydrogen phosphate, dihydrogen phosphate, thiosulfate, perchlorate, chlorate, chlorite, carbonate, bicarbonate, amide, cyanide, cyanate, thiocyanate, peroxide, hydroxide, permanganate, or any other suitable anion.

Alternatively, the mineral salt may comprise the mineral as an anion, and a cationic counterion. Preferably, the cationic counterion comprises only cationic moieties that will not substantially replace the target mineral in the amino acid complex. For example, when the target mineral is molybdenum, the mineral reactant can comprise a molybdate salt. As another example, when the target mineral is boron, the mineral reactant can comprise a borate salt. The cation species in such circumstances can comprise, for example, hydrogen, an alkali metal (lithium, sodium, potassium, rubidium, cesium, francium), an alkaline earth metal (beryllium, magnesium, calcium, strontium, barium, radium), or any other appropriate cation species. In one embodiment, the cation species comprises an alkali metal. More particularly, the cation species can comprise sodium in a specific embodiment.

Non-limiting examples of suitable mineral salts include zinc nitrate, zinc chlorate, zinc sulfate, zinc phosphate, zinc molybdate, zinc acetate, ferrous sulfate, ferrous chloride, ferric sulfate, ferric chloride, molybdenuym (II) chloride, molybdenum (III) chloride, copper (II) sulfate, copper (II) chloride, manganese (II) sulfate, potassium permanganate, boric acid, sodium borate (e.g., sodium tetraborate decahydrate, disodium octaborate tetrahydrate), and potassium chloride.

As specific examples, the mineral reactant can comprise zinc sulfate, copper (II) sulfate, manganese (II) sulfate, sodium borate (e.g., disodium octaborate), sodium molybdate, and iron (II) sulfate, or a combination thereof.

The skilled person will understand that the desired concentration of the mineral reactant in the reaction mixture may vary depending on the target mineral and the selected counterion. As non-limiting examples, the concentration of the mineral reactant in the reaction mixture may be at least about 1 percent by weight, at least about 2 percent by weight, at least about 5 percent by weight, at least about 10 percent by weight, at least about 15 percent by weight, or at least about 20 percent by weight.

Conversely, the concentration of the mineral reactant in the reaction mixture may be no greater than about 75 percent by weight, no greater than about 50 percent by weight, no greater than about 40 percent by weight, no greater than about 30 percent by weight, no greater than about 25 percent by weight, no greater than about 20 percent by weight, no greater than about 15 percent by weight, or no greater than about 10 percent by weight.

### Amino Acid

The amino acid may be selected as described in detail above.

As non-limiting examples, the amino acid concentration in the composition may be at least about 1 percent by weight, at least about 2 percent by weight, at least about 5 percent by weight, at least about 10 percent by weight, at least about 15 percent by weight, or at least about 20 percent by weight. Conversely, the concentration of the amino acid may be no greater than about 75 percent by weight, no greater than about 50 percent by weight, no greater than about 40 percent by weight, no greater than about 30 percent by weight, no greater than about 25 percent by weight, no greater than about 20 percent by weight, no greater than about 15 percent by weight, or no greater than about 10 percent by weight.

The skilled person will understand that the desired concentration of the amino acid may vary depending on the applicable amino acid. For example, when the amino acid is glycine, the concentration of the amino acid may be more particularly from about 1 percent by weight to about 25 percent by weight, from about 1 percent by weight to about 20 percent by weight, from about 2 percent by weight to about 20 percent by weight, from about 2 percent by weight to about 15 percent by weight, or from about 2 percent by weight to about 10 percent by weight. When the amino acid is lysine, the concentration of the amino acid may be more particularly from about 1 percent by weight to about 50 percent by weight, from about 1 percent by weight to about 40 percent by weight, from about 10 percent by weight to about 40 percent by weight, from about 10 percent by weight to about 30 percent by weight, or from about 10 percent by weight to about 25 percent by weight.

The molar ratio of the mineral to the amino acid in the reaction mixture is typically about 1:1, for example between about 0.5:1 and about 2:1, or between about 0.25:1 and about 4:1. The molar ratio of the mineral to the amino acid in the reaction mixture is preferably at least about 0.1:1, at least about 0.2:1, at least about 0.3:1, at least about 0.4:1, at least about 0.5:1, at least abut 0.6:1, at least about 0.7:1, at least about 0.8:1, or at least about 0.9:1. The molar ratio of the mineral to the amino acid in the reaction mixture is preferably no greater than about 10:1, no greater than about 9:1, no greater than about 8:1, no greater than about 7:1, no greater than about 6:1, no greater than about 5:1, no greater than about 4:1, no greater than about 3:1, or no greater than about 2:1.

### Reaction Procedure

The method may comprise an amino acid addition step wherein the amino acid is mixed with the solvent. Preferably, the amino acid is fully dissolved in the solvent and the reaction mixture forms a clear solution. As used herein, the terms “mixed” or “mixing” are intended to broadly encompass any method of physically combining two components, and do not require the use of mechanical agitation.

The method may further comprise a mineral reactant addition step wherein the mineral reactant is mixed with the solvent. Preferably, the mineral reactant addition step is carried out after the amino acid addition step, so that the mineral reactant is added to a reaction mixture comprising the solvent (e.g., water) and the amino acid. Alternatively, the mineral reactant addition step and the amino acid addition step may be carried out simultaneously.

Without being bound to a particular theory, it is currently believed that when a metal sulfate (or chloride) is dissolved in water, the positive and negative species dissociate into the solution. In the case of sulfates, it is believed that the sulfate ion (SO₄⁻) species reacts with water to create sulfuric acid (H₂SO₄), and the mineral element freely searches for an ionic bond. When an amino acid is introduced into the solution, it is believed that the hydroxyl group at the terminus end releases a hydrogen ion (H⁺) into solution, and the oxygen ion (O²⁻) forms a coordination bond between the free element (e.g., Zn⁺).

Again without being bound to a particular theory, it is currently believed that other minerals, such as boron, form an ionic association with one or more amino groups present on the amino acid, as discussed in further detail below.

The solvent may be heated during the amino acid addition step and/or during the mineral reactant addition step. Heat may be useful, for example, to (1) increase the rate of the reaction between the amino acid and the mineral reactant, and/or (2) to increase the solubility of the mineral reactant in the solvent. Preferably, heat is added in an amount sufficient to ensure the complete dissolution of the mineral reactant. Most preferably, heat is added to raise the temperature of the solvent to a value at or above the melting point of the mineral reactant.

When the amino acid is lysine, the reaction mixture is preferably maintained at a temperature of between about 50° C. and about 80° C., more preferably between about 65° C. and about 75° C. (e.g., about 70° C.). Without being bound to a particular theory, it is believed that this temperature is effective to maintain an aqueous solution of lysine and the mineral reactant, and significantly shortens the reaction time as compared to an otherwise similar room-temperature reaction.

### Exemplary Reaction Procedure for Boron Lysinate

As a non-limiting example, a reaction procedure suitable to prepare boron lysinate is described in detail below.

The reaction to create the boron lysinate is a two-step reaction. in the first step, disodium octaborate tetrahydrate is dissolved in an excess of water. The reaction in Step 1 is quantified in Equation 1:

$${Na}_{2} {B}_{8} {O}_{13} \cdot 4 {H}_{2} O + 9 {H}_{2} O {\to }^{yields} 2 NaOH + 8 {B ( OH )}_{3} \qquad Equation 1$$

When the disodium octaborate tetrahydrate is dissolved in excess water, it dissociates into boric acid and sodium hydroxide. The solution is mildly acidic with a pH of 6.6. As the dissolution is occurring as explained in Equation One, the solution is heated to about 70° C. (about 160° F.). Around this temperature, the boric acid loses a hydroxyl group to become metaboric acid. This reaction is explained by Equation 2 below.

$${B ( OH )}_{3} {\to }^{Δ H} {HBO}_{2} + {H}_{2} O \qquad Equation 2$$

Once the solution reaches 160° F., lysine is added to the solution. The lysine dissolves and when in a solution with a close to neutral pH, the two amino groups in lysine will exhibit a positive charge, as the amino groups will take on an extra hydrogen (Equation 3). The ability of the amino groups (alpha- and zeta-) in lysine to either accept or shed Hydrogen depending on the pH of the solution is what gives lysine its ability to create ionic bonds between other species in solution.

$${NH}_{2} ⇔ {NH}_{3}^{+} \qquad Equation 3$$

The zeta-linkage has a slightly stronger polarity (pKa=10.5 vs. pKa=9.0) compared to the alpha-linkage. However, both polarities at the solution pH of 6.6 are strong, especially when compared to saddle of the hydroxyl and carbonyl group. When the amino groups polarize in the mixing solution, the polar metaboric acid forms an ionic bond with either the alpha- or zeta-amino groups on lysine. When this bond is formed, the boron becomes neutrally charged, and is made highly available within the plant, allowing for translocation between the xylem and phloem through the spiral tubes. In contrast, boric acid not bound to lysine will only move between the two circulation systems when the pressure and polarity gradient of circulating boric acid ions allows for the transfer.

Additionally, and without being bound to a particular theory, it is believed that meta-boric acid in the presence of excess heat can form tetraboric acid ([B₄O₇]²⁻). The formation of tetraboric acid from metaboric acid could result in the formation of two ionic bonds, between both the alpha- and zeta-amino groups in a solution with close to neutral ph. The formation of the two ionic bonds would result in a traditional “claw” structure for the boron lysinate, which would be similar in conformation to a traditional Zn-EDTA. This structure is illustrated below as Formula IId.

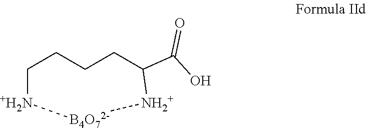

### Application to Plants, Soil, and Seeds

In various embodiments, the disclosure is generally related to providing a desired mineral to a plant. For example, in one embodiment, a treatment composition comprising an amino acid complex as described herein may be supplied to a plant exogenously. For example, the treatment composition may comprise a ready-to-use formulation as described above. Where a concentrate composition is used, the concentrate composition is first diluted to an agronomically acceptable concentration through addition of a solvent (e.g., water) to form a treatment composition.

The treatment composition may be applied to the plant and/or the surrounding soil through spray, drips, and/or other forms of liquid application. In another embodiment, a treatment composition comprising the complex is directly applied to the soil surrounding the root zone of a plant. The treatment composition can be applied as an aqueous solution, an emulsion, a suspension, a granular composition, or a powder, as appropriate.

The application may be performed using any method or apparatus known in the art, including but not limited to hand sprayer, mechanical sprinkler, or irrigation, including drip irrigation. For example, in one embodiment, the composition is applied to plants and/or soil using a drip irrigation technique. The treatment composition is preferably applied directly to the base of the plants or the soil immediately adjacent to the plants. This procedure is particularly preferred for use in connection with cotton, strawberries, tomatoes, potatoes, vegetables, and ornamental plants.

In another embodiment, the amino acid complex composition is applied to plants and/or soil using a drench application. Typically, a sufficient quantity of the treatment composition is applied such that it drains through the soil to the root area of the plants. The drench application technique is particularly preferred for use in connection with crop plants and turf grasses.

In various embodiments, the treatment composition is applied to soil after planting. In other embodiments, the treatment composition may be applied to soil during planting. In yet further embodiments, the treatment composition may be applied to soil before planting. Thus, the treatment composition may be applied to soil before, during, or after planting, or any combination thereof. When the composition is applied directly to the soil, it may be applied using any method known in the art. For example, it may be tilled into the soil or applied in furrow.

Generally, the compounds described herein can be applied to seeds, plants, or the environment of plants in order to aid in mineral absorption. For example, in one embodiment, the method comprises administering to a plant, a seed, or soil a composition comprising an effective amount of the amino acid complex as described herein. The term “exogenous application” is intended to refer to any application method that causes the application composition to come into contact with the plant, plant part, or plant seed and includes any of the methods described above, including application to the soil or the area surrounding the plant, plant part, or plant seed.

The term “effective amount” will be readily understood by the skilled person and means application of an amount delivering a sufficient concentration of the amino acid complex in order to allow for an agriculturally sufficient amount of the complex to be readily available to the plant or plant part for absorption.

The present disclosure also provides a method for improving the vigor of a plant as compared with an untreated plant comprising administering to a plant, a seed, or soil a composition comprising an effective amount of the amino acid complex. The improved vigor may comprise increased crop productivity. The increased crop productivity can, in some circumstances, comprise increased yield, increased plant parts or storage organs, increased water function, increased stress tolerance, increased protection against an abiotic stressor, enhanced phenotypic characteristics, increased protection against herbicide injury, increased efficacy of an herbicide, improved maintenance of the health and vigor of flower, increased growth rate, or a combination of any thereof.

The increased water function can comprise increased water movement into and through the plant, greater water retention, increased water-use efficiency, increased turgor, or a combination of any thereof.

The abiotic stressor can comprise high temperatures, such as temperatures above 29° C., low temperatures, such as temperatures below 12° C., water deficit, drought, desiccation, high humidity, such as humidity above 60%, low humidity, such as humidity below 30%, fluctuations in humidity, osmotic fluctuations, high salinity, increased transpiration, low soil moisture, UV stress, radiation stress, or a combination of any thereof.

Enhanced phenotypic characteristics can comprise increased chlorophyll, increased duration for greenness, reduced senescence, increased turgor, enhanced plant growth and appearance, prevention of chlorosis, prevention of stunted growth, prevention of leaf rolling, preventing of leaf curling, prevention of leaf, floral, and/or fruit abscission, or a combination of any thereof.

The composition and methods described herein can be used in connection with any species of plant and/or the seeds thereof. The compositions and methods are typically used in connection with seeds that are agronomically important. The seed can be a transgenic seed from which a transgenic plant can grow that incorporates a transgenic event that confers, for example, tolerance to a particular herbicide or combination of herbicides, increased disease resistance, enhanced tolerance to insects, drought, stress and/or enhanced yield. The seed can comprise a breeding trait, including for example, a disease tolerant breeding trait. In some instances, the seed includes at least one transgenic trait and at least one breeding trait.

The compositions and methods can be used for the treatment of any suitable seed type, including, but not limited to row crops and vegetables. For example, one or more plants or plant parts or the seeds of one or more plants can comprise abaca (manila hemp) (Musa textilis), alfalfa for fodder (Medicago sativa), alfalfa for seed (Medicago sativa), almond (Prunus dulcis), anise seeds (Pimpinella anisum), apple (Malus sylvestris), apricot (Prunus armeniaca), areca (betel nut) (Areca catechu), arracha (Arracacia xanthorrhiza), arrowroot (Maranta arundinacea), artichoke (Cynara scolymus), asparagus (Asparagus officinalis), avocado (Persea americana), bajra (pearl millet) (Pennisetum americanum), bambara groundnut (Vigna subterranea), banana (Musa paradisiaca), barley (Hordeum vulgare), beans, dry, edible, for grains (Phaseolus vulgaris), beans, harvested green (Phaseolus and Vigna spp.), beet, fodder (mangel) (Beta vulgaris), beet, red (Beta vulgaris), beet, sugar (Beta vulgaris), beet, sugar for fodder (Beta vulgaris), beet, sugar for seeds (Beta vulgaris), bergamot (Citrus bergamia), betel nut (Areca catechu), black pepper (Piper nigrum), black wattle (Acacia mearnsii), blackberries of various species (Rubus spp.), blueberry (Vaccinium spp.), Brazil nut (Bertholletia excelsa), breadfruit (Artocarpus altilis), broad bean, dry (Vicia faba), broad bean, harvested green (Vicia faba), broccoli (Brassica oleracea var. botrytis), broom millet (Sorghum bicolor), broom sorghum (Sorghum bicolor), Brussels sprouts (Brassica oleracea var. gemmifera), buckwheat (Fagopyrum esculentum), cabbage, red, white, Savoy (Brassica oleracea var. capitata), cabbage, Chinese (Brassica chinensis), cabbage, for fodder (Brassica spp.), cacao (cocoa) (Theobroma cacao), cantaloupe (Cucumis melo), caraway seeds (Carum carvi), cardamom (Elettaria cardamomum), cardoon (Cynara cardunculus), carob (Ceratonia siliqua), carrot, edible (Daucus carota spp. sativa), carrot, for fodder (Daucus carota sativa), cashew nuts (Anacardium occidentale), cassava (manioc) (Manihot esculenta), castor bean (Ricinus communis), cauliflower (Brassica oleracea var. botrytis), celeriac (Apium graveolens var. rapaceum), celery (Apium graveolens), chayote (Sechium edule), cherry, all varieties (Prunus spp.), chestnut (Castanea sativa), chickpea (gram pea) (Cicer arietinum), chicory (Cichorium intybus), chicory for greens (Cichorium intybus), chili, dry (all varieties) (Capsicum spp. (annuum)), chili, fresh (all varieties) (Capsicum spp. (annuum)), cinnamon (Cinnamomum verum), citron (Citrus medica), citronella (Cymbopogon citrates; Cymbopogon nardus), clementine (Citrus reticulata), clove (Eugenia aromatica; Syzygium aromaticum), clover for fodder (all varieties) (Trifolium spp.), clover for seed (all varieties) (Trifolium spp.), cocoa (cacao) (Theobroma cacao), coconut (Cocos nucifera), cocoyam (Colocasia esculenta), coffee (Coffea spp.), cola nut, all varieties (Cola acuminata), colza (rapeseed) (Brassica napus), corn (maize), for cereals (Zea mays), corn (maize), for silage (Zea mays), corn (maize), for vegetable (Zea mays), corn for salad (Valerianella locusta), cotton, all varieties (Gossypium spp.), cottonseed, all varieties (Gossypium spp.), cowpea, for grain (Vigna unguiculata), cowpea, harvested green (Vigna unguiculata), cranberry (Vaccinium spp.), cress (Lepidium sativum), cucumber (Cucumis sativus), currants, all varieties (Ribes spp.), custard apple (Annona reticulate), dasheen (Colocasia esculenta), dates (Phoenix dactylifera), drumstick tree (Moringa oleifera), durra (sorghum) (Sorghum bicolour), durum wheat (Triticum durum), earth pea (Vigna subterranea), edo (eddoe) (Xanthosoma spp.; Colocasia spp.), eggplant (Solanum melongena), endive (Cichorium endivia), fennel (Foeniculum vulgare), fenugreek (Trigonella foenum-graecum), fig (Ficus carica), filbert (hazelnut) (Corylus avellana), fique (Furcraea macrophylla), flax for fiber (Linum usitatissimum), flax for oil seed (linseed) (Linum usitatissimum), formio (New Zealand flax) (Phormium tenax), garlic, dry (Allium sativum), garlic, green (Allium sativum), geranium (Pelargonium spp.; Geranium spp.), ginger (Zingiber officinale), gooseberry, all varieties (Ribes spp.), gourd (Lagenaria spp; Cucurbita spp.), gram pea (chickpea) (Cicer arietinum), grape (Vitis vinifera), grapefruit (Citrus paradisi), grapes for raisins (Vitis vinifera), grapes for table use (Vitis vinifera), grapes for wine (Vitis vinifera), grass esparto (Lygeum spartum), grass, orchard (Dactylis glomerata), grass, Sudan (Sorghum bicolor var. sudanense), groundnut (peanut) (Arachis hypogaea), guava (Psidium guajava), guinea corn (sorghum) (Sorghum bicolor), hazelnut (filbert) (Corylus avellana), hemp fiber (Cannabis sativa spp. indica), hemp, manila (abaca) (Musa textilis), hemp, sun (Crotalaria juncea), hempseed (marijuana) (Cannabis sativa), henequen (Agave fourcroydes), henna (Lawsonia inermis), hop (Humulus lupulus), horse bean (Vicia faba), horseradish (Armoracia rusticana), hybrid maize (Zea mays), indigo (Indigofera tinctoria), jasmine (Jasminum spp.), Jerusalem artichoke (Helianthus tuberosus), jowar (sorghum) (Sorghum bicolor), jute (Corchorus spp.), kale (Brassica oleracea var. acephala), kapok (Ceiba pentandra), kenaf (Hibiscus cannabinus), kohlrabi (Brassica oleracea var. gongylodes), lavender (Lavandula spp.), leek (Allium ampeloprasum; Allium porrum), lemon (Citrus limon), lemongrass (Cymbopogon citratus), lentil (Lens culinaris), lespedeza, all varieties (Lespedeza spp.), lettuce (Lactuca sativa var. capitata), lime, sour (Citrus aurantifolia), lime, sweet (Citrus limetta), linseed (flax for oil seed) (Linum usitatissimum), licorice (Glycyrrhiza glabra), litchi (Litchi chinensis), loquat (Eriobotrya japonica), lupine, all varieties (Lupinus spp.), Macadamia (Queensland nut) (Macadamia spp. ternifolia), mace (Myristica fragrans), maguey (Agave atrovirens), maize (corn) (Zea mays), maize (corn) for silage (Zea mays), maize (hybrid) (Zea mays), maize, ordinary (Zea mays), mandarin (Citrus reticulata), mangel (fodder beet) (Beta vulgaris), mango (Mangifera indica), manioc (cassava) (Manihot esculenta), maslin (mixed cereals) (mixture of Triticum spp. and Secale cereale), medlar (Mespilus germanica), melon, except watermelon (Cucumis melo), millet broom (Sorghum bicolor), millet, bajra (Pennisetum americanum), millet, bulrush (Pennisetum americanum), millet, finger (Eleusine coracana), millet, foxtail (Setaria italica), millet, Japanese (Echinochloa esculenta), millet, pearl (bajra, bulrush) (Pennisetum americanum), millet, proso (Panicum miliaceum), mint, all varieties (Mentha spp.), mulberry for fruit, all varieties (Morus spp.), mulberry for silkworms (Morus alba), mushrooms (Agaricus spp.; Pleurotus spp.; Volvariella), mustard (Brassica nigra; Sinapis alba), nectarine (Prunus persica var. nectarina), New Zealand flax (formio) (Phormium tenax), Niger seed (Guizotia abyssinica), nutmeg (Myristica fragrans), oats, for fodder (Avena spp.), oil palm (Elaeis guineensis), okra (Abelmoschus esculentus), olive (Olea europaea), onion seed (Allium cepa), onion, dry (Allium cepa), onion, green (Allium cepa), opium (Papaver somniferum), orange (Citrus sinensis), orange, bitter (Citrus aurantium), ornamental plants (various), palm palmyra (Borassus flabelfer), palm, kernel oil (Elaeis guineensis), palm, oil (Elaeis guineensis), palm, sago (Metroxylon sagu), papaya (pawpaw) (Carica papaya), parsnip (Pastinaca sativa), pea, edible dry, for grain (Pisum sativum), pea, harvested green (Pisum sativum), peach (Prunus persica), peanut (groundnut) (Arachis hypogaea), pear (Pyrus communis), pecan nut (Carya illinoensis), pepper, black (Piper nigrum), pepper, dry (Capsicum spp.), persimmon (Diospyros kaki; Diospyros virginiana), pigeon pea (Cajanus cajan), pineapple (Ananas comosus), pistachio nut (Pistacia vera), plantain (Musa sapientum), plum (Prunus domestica), pomegranate (Punica granatum), pomelo (Citrus grandis), poppy seed (Papaver somnferum), potato (Solamum tuberosum), palm, kernel oil (Elaeis guineensis), potato, sweet (Ipomoea batatas), prune (Prunus domestica), pumpkin, edible (Cucurbita spp.), pumpkin, for fodder (Cucurbita spp.), pyrethum (Chrysanthemum cinerariaefolium), quebracho (Aspidosperma spp.), Queensland nut (Macadamia spp. ternifolia), quince (Cydonia oblonga), quinine (Cinchona spp.), quinoa (Chenopodium quinoa), ramie (Boehmeria nivea), rapeseed (colza) (Brassica napus), raspberry, all varieties (Rubus spp.), red beet (Beta vulgaris), redtop (Agrostis spp.), rhea (Boehmeria nivea), rhubarb (Rheum spp.), rice (Oryza sativa; Oryza glaberrima), rose (Rose spp.), rubber (Hevea brasiliensis), rutabaga (swede) (Brassica napus var. napobrassica), rye (Secale cereale), ryegrass seed (Lolium spp.), safflower (Carthamus tinctorius), sainfoin (Onobrychis viciifolia), salsify (Tragopogon porrifolius), sapodilla (Achras sapota), satsuma (mandarin/tangerine) (Citrus reticulata), scorzonera (black salsify) (Scorzonera hispanica), sesame (Sesamum indicum), shea butter (nut) (Vitellaria paradoxa), sisal (Agave sisalana), sorghum (Sorghum bicolor), sorghum, broom (Sorghum bicolor), sorghum, durra (Sorghum bicolor), sorghum, guinea corn (Sorghum bicolor), sorghum, jowar (Sorghum bicolor), sorghum, sweet (Sorghum bicolor), soybean (Glycine max), soybean hay (Glycine max), spelt wheat (Triticum spelta), spinach (Spinacia oleracea), squash (Cucurbita spp.), strawberry (Fragaria spp.), sugar beet (Beta vulgaris), sugar beet for fodder (Beta vulgaris), sugar beet for seed (Beta vulgaris), sugarcane for fodder (Saccharum officinarum), sugarcane for sugar or alcohol (Saccharum officinarum), sugarcane for thatching (Saccharum officinarum), sunflower for fodder (Helianthus annuus), sunflower for oil seed (Helianthus annuus), sunhemp (Crotalaria juncea), swede (Brassica napus var. napobrassica), swede for fodder (Brassica napus var. napobrassica), sweet corn (Zea mays), sweet lime (Citrus limetta), sweet pepper (Capsicum annuum), sweet potato (Lopmoea batatas), sweet sorghum (Sorghum bicolor), tangerine (Citrus reticulata), tannia (Xanthosoma sagittifolium), tapioca (cassava) (Manihot esculenta), taro (Colocasia esculenta), tea (Camellia sinensis), teff (Eragrostis abyssinica), timothy (Phleum pratense), tobacco (Nicotiana tabacum), tomato (Lycopersicon esculentum), trefoil (Lotus spp.), triticale, for fodder (hybrid of Triticum aestivum and Secale cereale), tung tree (Aleurites spp.; Fordii), turnip, edible (Brassica rapa), turnip, for fodder (Brassica rapa), urena (Congo jute) (Urena lobata), vanilla (Vanilla planfolia), vetch, for grain (Vicia sativa), walnut (Juglans spp., especially Juglans regia), watermelon (Citrullus lanatus), wheat (Triticum aestivum), yam (Dioscorea spp.), yerba mate (Ilex paraguariensis).

The compositions and methods disclosed herein can also be applied to turf grass, ornamental grass, flowers, ornamentals, trees, and shrubs. The agricultural compositions are also suitable for use in the nursery, lawn, and garden, floriculture or the cut flower industry and provides certain benefits. For example, the compositions can be applied to perennials, annuals, forced bulbs, or pseudo bulbs, herbs, groundcovers, trees, shrubs, ornamentals (e.g., orchids, etc.), tropicals, and nursery stock.

The methods described herein can comprise applying to a seed of a plant (e.g., any plant herein described) a treatment composition. The seed treatment method can comprise applying the composition to the seed prior to sowing the seed, so that the sowing operation is simplified. The composition can also be applied to seeds by any standard seed treatment methodology, including but not limited to mixing in a container, mechanical application, tumbling, spraying, immersion, and solid matrix priming. Any conventional active or inert material can be used for contacting seeds with the composition, such as conventional film-coating materials including but not limited to water-based film coating materials.

### Methods of Treating Iron Chlorosis

In the methods, compositions, and mineral:amino acid complexes described above, the mineral:amino acid complex can comprise a combination of iron lysinate and magnesium lysinate. Alternatively, the methods, compositions, and mineral:amino acid complexes described above, the mineral:amino acid complex can comprise a combination of iron lysinate and manganese lysinate.

In these embodiments, the methods, compositions, and mineral:amino acid complexes are suitable for treating iron chlorosis present in crop plants (e.g., corn plants or soybean plants).

The use of iron and either magnesium or manganese lysinate blends transports both iron and magnesium/manganese to the leaf where the production of chlorophyll can be restarted. Iron chlorosis is typically caused by insufficient production of chlorophyll. Chlorophyll is important in the process for production of phytosideraphore, which is responsible for reducing soil iron. Reduced iron, in turn, is necessary for the production of chlorophyll, causing a feedback loop when iron chlorosis sets in. The use of the mineral lysinate blends, and specifically iron and magnesium lysinate, solves this problem by providing readily available essential iron and magnesium to the plant so that the production of chlorophyll can be restarted. This also causes the production of phytosideraphore to be restarted. Once the feedback loop is fixed, iron reduction processes can be recovered.

For example, provided herein is a method of treating iron chlorosis in a plant, the method comprising applying to a plant an effective amount of an agrochemical composition comprising (1) iron lysinate, and (2) magnesium lysinate, manganese lysinate, or a combination thereof.

Also provided herein is an aqueous agrochemical composition useful for treating iron chlorosis. For example, the composition may comprise a first mineral lysinate complex of Formula Ia wherein M is iron; a second mineral lysinate complex of Formula Ia wherein M is manganese or magnesium; and water. Preferably, in the second mineral lysinate complex, M is magnesium. The ratio of the first mineral lysinate complex to the second mineral lysinate complex is preferably between about 1:4 and about 4:1, for example between about 1:3 and about 3:1, or between about 1:2 and about 2:1, or about 1:1 on a molar basis.

The composition may be a ready-to-use composition or a concentrate composition wherein the concentration of the mineral lysinate complex is selected as described in detail above. The composition may further comprise one or more excipients a s described in detail above.

The term “effective amount” will be readily understood by the skilled person and means application of an amount delivering a sufficient concentration of the mineral:amino acid complex in order to allow for an agriculturally sufficient amount of the complex to be readily available to the plant or plant part for absorption. Typically, the amount of the composition applied in order to treat or reduce iron chlorosis may be less than an amount needed using conventional chelating agents, such as EDTA or EDDHA. For example, depending on the concentration of a ready-to-use formulation, about 1 to 5 gallons per acre of the composition is applied. Preferably, about 1 to 3 gallons per acre is applied.

As a non-limiting example, to treat iron chlorosis or for the other purposes described herein, the mineral:amino acid complex may applied to crop plants at a rate of at least about 50 grams of the mineral lysinate complex per acre, for example a rate of at least about 100 grams per acre, at least about 200 grams per acre, at least about 300 grams per acre, at least about 400 grams per acre, at least about 500 grams per acre, at least about 1 kilogram per acre, at least about 2 kilograms per acre, at least about 3 kilograms per acre, or at least about 4 kilograms per acre. The skilled person will appreciate that the optimal application rate will vary based upon the mineral species to be supplied to the plant, and the plant's condition at the time of application (e.g., whether the plant is deficient in the mineral species to be supplied, and if so, to what degree).

The present disclosure also provides a method for improving the vigor of a plant as compared with an untreated plant comprising administering to a plant, a seed, or soil a composition comprising an effective amount of the mineral:amino acid complex. The improved vigor may comprise increased crop productivity. The increased crop productivity can, in some circumstances, comprise increased yield, increased plant parts or storage organs, increased water function, increased stress tolerance, increased protection against an abiotic stressor, enhanced phenotypic characteristics, increased protection against herbicide injury, increased efficacy of an herbicide, improved maintenance of the health and vigor of flower, increased growth rate, or a combination of any thereof.

The increased water function can comprise increased water movement into and through the plant, greater water retention, increased water-use efficiency, increased turgor, or a combination of any thereof.

The abiotic stressor can comprise high temperatures, such as temperatures above 29° C., low temperatures, such as temperatures below 12° C., water deficit, drought, desiccation, high humidity, such as humidity above 60%, low humidity, such as humidity below 30%, fluctuations in humidity, osmotic fluctuations, high salinity, increased transpiration, low soil moisture, UV stress, radiation stress, or a combination of any thereof.

Enhanced phenotypic characteristics can comprise increased chlorophyll, increased duration for greenness, reduced senescence, increased turgor, enhanced plant growth and appearance, prevention of chlorosis, prevention of stunted growth, prevention of leaf rolling, preventing of leaf curling, prevention of leaf, floral, and/or fruit abscission, or a combination of any thereof.

The composition and methods described herein can be used in connection with any species of plant and/or the seeds thereof. The compositions and methods are typically used in connection with seeds that are agronomically important. The seed can be a transgenic seed from which a transgenic plant can grow that incorporates a transgenic event that confers, for example, tolerance to a particular herbicide or combination of herbicides, increased disease resistance, enhanced tolerance to insects, drought, stress and/or enhanced yield. The seed can comprise a breeding trait, including for example, a disease tolerant breeding trait. In some instances, the seed includes at least one transgenic trait and at least one breeding trait.

As used herein, the term “alkylene” refers to both straight and branched chain radicals having from about 1 to about 22 carbon atoms, unless otherwise specified, which may be optionally independently substituted. Non-limiting examples of alkylene groups include methylene, ethylene, propylene, isopropylene, butylene, sec-butylene, tert-butylene, 3-pentylene, hexylene, and octylene groups, each of which may be optionally independent substituted.

The term “substituted” as in “substituted alkyl,” and the like, means that in the group in question (i.e., the alkyl, aryl or other group that follows the term), at least one hydrogen atom bound to a carbon atom is replaced with one or more substituent groups such as hydroxy (—OH), alkylthio, phosphino, amido (—CON(R_{A})(R_{B})), wherein R_{A} and R_{B} are independently hydrogen, alkyl, or aryl), amino (—N(R_{A})(R_{B})), wherein R_{A} and R_{B} are independently hydrogen, alkyl, or aryl), halo (fluoro, chloro, bromo, or iodo), silyl, nitro (—NO₂), an ether (—OR_{A} wherein R_{A} is alkyl or aryl), an ester (—OC(O)R_{A} wherein R_{A} is alkyl or aryl), keto (—C(O)R_{A} wherein R_{A} is alkyl or aryl), heterocyclo, and the like. When the term “substituted” introduces a list of possible substituted groups, it is intended that the term apply to every member of that group. That is, the phrase “optionally substituted alkyl or aryl” is to be interpreted as “optionally substituted alkyl or optionally substituted aryl.”

### EXAMPLES

The following non-limiting examples are provided to further illustrate the present invention.

### Example 1: Synthesis of Glycinate Compositions

Table 1 reports the chemical properties of the various ingredients and components utilized in this Example. As discussed, glycinates are chemically classified as coordination complexes, and the reaction between the mineral element (“element”) and the amino acid (“AA”) is stoichiometrically balanced by determining the ratio between the amino acid molecular weight and the molecular weight of the element in solution. This relationship is mathematically depicted in the equation:

$$Molar Ratio = \frac{M . W . AA}{M . W . Element}$$

TABLE 1
Chemical composition of test ingredients.

| | | | |
|---|---|---|---|
| | Element Conc. | Elemental MW | Molar Ratio |
| Ingredient | (weight %) | (g/mol) | (AA:Element) |

Minerals

| | | | |
|---|---|---|---|
| ZnSO₄ | 35.5 | 65 | 1.15 |
| CuSO₄ | 25.5 | 63 | 1.19 |
| MnSO₄ | 32.0 | 55 | 1.37 |
| Sodium borate | 22.5 | 11 | 6.94 |
| Sodium molybdate | 39.4 | 96 | 0.78 |
| FeSO₄ | 32.3 | 56 | 1.34 |
| KCl | 51.5 | 39 | 1.92 |

Amino Acids

| | | | |
|---|---|---|---|
| Glycine | >99 | 75 | — |

The test formulas for the experiment are listed in Table 2. Deriving the formulas required two steps: (1) dissolving a mineral sulfate in tap water; and (2) reacting the disassociated mineral element with the amine acid (in this Example, glycine). The initial elemental concentrations were determined using a commercial market survey. Using zinc 8.5% (Table 2) as an example, the following calculations were completed:

$${ZnSO}_{4} = \frac{S o l ution Concentration}{Elem ental Concentration} = \frac{8.5}{0.355}$$

This calculation effectively determined the amount of mineral sulfate required. Following this calculation, the amount of amino acid required was calculated as follows:

$$Amino Acid = Soln . Conc . \times Molar Ratio = 8.5 \times 1.15$$

TABLE 2
Test formulations.

| | | | Mineral | | | |
|---|---|---|---|---|---|---|
| | | Mineral | reactant | Glycine | Water | |
| Solution | Mineral | reactant | (weight %) | (weight %) | (weight %) | Total |
| G1 | Zinc 8.5% | ZnSO₄ | 24.0 | 10.0 | 66.0 | 100.0 |
| G2 | Copper 6.0% | CuSO₄ | 24.0 | 7.0 | 69.0 | 100.0 |
| G3 | Manganese 6.0% | MnSO₄ | 19.0 | 8.0 | 73.0 | 100.0 |
| G4 | Iron 3.0% | FeSO₄ | 9.0 | 4.0 | 87.0 | 100.0 |
| G5 | Boron 3.5% | Sodium | 16.0 | 24.0 | 60.0 | 100.0 |
| | | borate | | | | |
| G6 | Molybdenum | Sodium | 2.0 | 3.0 | 95.0 | 100.0 |
| | 0.5% | molybdate | | | | |
| G7 | Potassium 5% | KCl | 9.7 | 9.6 | 80.7 | 100.0 |

In order to create the solutions provided in Table 2, tap water was first placed into a 500-mL beaker, and the beaker was placed on a hot plate with a magnetic stirrer. A stir bar was then placed inside the beaker and the magnetic stirrer was turned to setting 8. A thermocouple was also inserted into the water in order to measure the temperature of the water. Once the initial temperature was measured, the mineral reactant was poured into the water and a stopwatch was started. The temperature of the solution was measured at 30-second increments. Upon dissolution of the mineral reactant, the amino acid (in this Example, glycine) was added to the solution and the temperature and time of addition were recorded. The end point of the coordination reaction was determined to be the point when (a) all solute was dissolved and (b) the temperature of the solution stabilized.

Thus, the measured variables included (1) the mineral temperature maximum/minimum (T_{M}); (2) the amino acid temperature maximum/minimum (T_{AA}); (3) the amino acid addition time; and (4) the reaction completion time. These variables for glycinate solutions G1 to G7 are reported in Table 3.

TABLE 3
Reaction variables.

| | | | AA | Reaction |
|---|---|---|---|---|
| Solution | T_{M} (° C. (° F.)) | T_{AA} (° C. (° F.)) | addition (s) | end (s) |
| G1 | 41.8 (107.2) | 28.7 (83.7) | 840 | 1030 |
| G2 | 23.9 (75.1) | 18.0 (64.4) | 240 | 510 |
| G3 | 33.1 (91.5) | 27.4 (81.4) | 270 | 370 |
| G4 | 27.7 (81.8) | 25.2 (77.3) | 470 | 810 |
| G7 | 23.3 (74.0) | 14.6 (58.2) | 340 | 840 |

This Example tested the initial formulas, formulated by determining the ratio between the molecular weight of glycine and of the target mineral element. This ratio assumes complete dissolution of the salt and the subsequent free availability of the mineral element. For solutions containing zinc, manganese, and iron (solutions G1, G3, and G4, respectively), there was an increase in the solution temperature as the salt was dissolved. For solutions containing copper and potassium (solutions G2 and G7, respectively), there was a decrease in the solution temperature as the salt was dissolved. In all cases, there was a sharp temperature depression upon addition of the glycine to the dissolved salt solution. It is foreseen that these temperature profiles can be used to code the logic to control the production cycles of each solution using a programmable logic controller (PLC).

It was found that using glycine as the amino acid resulted in an irreversible precipitation of the boron salt after the reaction reached its terminus. Without being bound to a particular theory, it is theorized that the precipitation is attributed to only one proton being available to complex with glycine, which has two available electrons.

### Example 2: Synthesis of Lysinate Solutions

Table 4 reports the chemical properties of the various ingredients and components utilized in this Example. As discussed, lysine, an amino acid, forms a coordination complex with a disassociated mineral element. The same equations described in Example 1 are also used in this Example to determine elemental concentration, molecular weight, and the molar ratio of the amino acid to the element.

TABLE 4
Chemical composition of test ingredients.

| | | | |
|---|---|---|---|
| | Element Conc. | Elemental MW | Molar Ratio |
| Ingredient | (weight %) | (g/mol) | (AA:Element) |

Minerals

| | | | |
|---|---|---|---|
| ZnSO₄ | 35.5 | 65 | 2.25 |
| CuSO₄ | 25.5 | 63 | 2.32 |
| MnSO₄ | 32.0 | 55 | 2.66 |
| Sodium borate | 21.5 | 11 | 13.52 |
| Sodium molybdate | 39.4 | 96 | 1.52 |
| FeSO₄ | 32.3 | 56 | 2.62 |
| KCl | 51.5 | 39 | 3.74 |

Amino Acids

| | | | |
|---|---|---|---|
| Lysine | >78 | 147 | — |

The test formulas for this Example are listed in Table 5.

TABLE 5
Test formulations.

| | | | Mineral | | | |
|---|---|---|---|---|---|---|
| | | Mineral | reactant | Lysine | Water | |
| Solution | Mineral | reactant | (weight %) | (weight %) | (weight %) | Total |
| L1 | Zinc 8.5% | ZnSO₄ | 24.0 | 24.0 | 52.0 | 100.0 |
| L2 | Copper 6.0% | CuSO₄ | 24.0 | 18.0 | 59.0 | 100.0 |
| L3 | Manganese 6.0% | MnSO₄ | 19.0 | 20.0 | 61.0 | 100.0 |
| L4 | Iron 3.0% | FeSO₄ | 9.0 | 10.0 | 81.0 | 100.0 |
| L5 | Boron 3.5% | Sodium | 14.0 | 25.3 | 60.7 | 100.0 |
| | | borate | | | | |
| L6 | Molybdenum 0.5% | Sodium | 2.0 | 9.0 | 89.0 | 100.0 |
| | | molybdate | | | | |
| L7 | Potassium 5% | KCl | 10.0 | 24.0 | 66.0 | 100.0 |

The same procedure as outlined in Example 1 was followed in this Example to create the solutions of Table 5, except that lysine was added as the amino acid in place of glycine. The measured variables included (1) the mineral temperature maximum/minimum (T_{M}); (2) the amino acid temperature maximum/minimum (T_{AA}); (3) the amino acid addition time; and (4) the reaction completion time. These variables for lysinate solutions L1 to L7 are reported in Table 6.

TABLE 6
Reaction Variables.

| | | | AA | Reaction |
|---|---|---|---|---|
| Solution | T_{M} (° C. (° F.)) | T_{AA} (° C. (° F.)) | addition (s) | end (s) |
| L1 | 47.3 (117.2) | 29.1 (84.4) | 410 | 910 |
| L2 | 21.3 (70.3) | 17.7 (63.9) | 240 | 510 |
| L3 | 34.5 (94.1) | 27.8 (82.0) | 210 | 670 |
| L4 | 28.1 (82.5) | −1.8 (28.7) | 310 | 610 |
| L5* | 57.5 (135.5) | 57.3 (135.1) | 440 | 1440 |
| L7 | 23.4 (74.1) | 15.9 (60.7) | 270 | 670 |

*Solution temperature was maintained at a constant heat setting of 54.4° C. (130° F.) for the entire reaction.

As can be seen from Table 6, the zinc:lysine reaction (solution L1) was the second longest in duration, which is primarily driven by the amount of time required to dissolve the zinc sulfate in water. The dissolution of the zinc sulfate in water resulted in the greatest temperature rise compared to the other tested minerals.

The manganese:lysine reaction (solution L3) had the second greatest temperature rise, but the manganese sulfate dissolution time was shorter than the zinc sulfate dissolution time. With regard to solution L4, there was a moderate temperature rise when the iron sulfate was dissolved in water. Conversely, there was a moderate temperature depression when the lysine was added to the mineral:water solution. The total reaction time was shorter than the zinc:lysine reaction time.

Referring to solution L2, there was a slight temperature depression upon addition of the copper sulfate to water. The temperature decreased further once lysine was added. This represented the shortest of all reactions.

Turning to solution L7, the dissolution of potassium chloride into the water resulted in the greatest temperature depression. The temperature decreased further once lysine was added. This represented the second longest reaction of all tested.

The boron solution (solution L5) was set at a hot plate setting sufficient to maintain a temperature of 54.4° C. (130° F.). The solution experienced a temperature drop upon addition of the sodium borate to the water. The dissolution time for the sodium borate was slightly longer than the dissolution time for zinc sulfate. Furthermore, the amount of time required for this coordination reaction to complete was long (1440 seconds). Additionally, the speed of the stir bar was substantially greater compared to other solutions (requiring a setting of 9 versus a setting of 5 for other reactions). For scale up purposes, the lysine should be metered in over a span of time to maintain a fluid state so that the reaction can drive to completion. A complete boron:lysine solution was ultimately achieved.

The zinc, copper, manganese, iron, and potassium reactions (L1 to L4 and L7, respectively) were very similar to the corresponding glycine reactions (G1 to G4 and G7, respectively). Lysine has a greater molecular weight as compared to glycine, so the amount of lysine required to drive the reaction to completion is greater compared to the glycine reactions. However, the mixing process was very similar to the glycine solutions.

It was found that the boron solution should be maintained at a minimum of 54.4° C. (130° F.) throughout the reaction to ease the dissolution of the sodium borate and to help catalyze the reaction between the lysine and the elemental boron.

### Example 3. Comparison of Liquid and Dry Lysine Formulations

Table 7 reports the chemical properties of the various ingredients and components utilized in this Example.

TABLE 7
Chemical composition of test ingredients.

| | | | |
|---|---|---|---|
| | Element Conc. | Elemental MW | Molar Ratio |
| Ingredient | (weight %) | (g/mol) | (AA:Element) |

Minerals

| | | | |
|---|---|---|---|
| SOLUBOR* | 20.5 | 11 | 13.52 |

Amino Acids

| | | | |
|---|---|---|---|
| Dry lysine | >78 | 147 | — |
| Liquid lysine | 55 | 147 | — |

*Disodium octaborate tetrahydrate (Na₂B₈O₁₃•4H₂O)

The test formulas for this Example are listed in Table 8.

TABLE 8
Test formulations.

| | | | Mineral | | | |
|---|---|---|---|---|---|---|
| Solution | | Mineral | reactant | Lysine | Water | |
| Type | Mineral | reactant | (weight %) | (weight %) | (weight %) | Total |
| Dry lysine | Boron 3.0% | SOLUBOR | 14.0 | 25.3 | 60.7 | 100.0 |
| Liquid lysine | Boron 3.0% | SOLUBOR | 14.0 | 37.0 | 49.0 | 100.0 |

To mix the solutions, tap water was first placed into a 500-mL beaker, and the beaker was placed on a hot plate with a magnetic stirrer. A stir bar was added to the beaker, and the magnetic stirrer was turned to setting 8. A thermocouple was also inserted into the water in order to measure the temperature of the water. The sodium borate was added to the beaker and a timer was started. The temperature was measured in 30-second increments. When all of the sodium borate dissolved, lysine was added and the temperature and time of the addition were recorded. The end point of the coordination reaction was recorded as the point when (1) all solute was dissolved and (2) the temperature of the solution stabilized. The viscosity at room temperature, pH, density, and boron concentration of the resulting solutions were measured. Additional measured variables included (1) the mineral temperature maximum/minimum (T_{M}); (2) the amino acid temperature maximum/minimum (T_{AA}); (3) the amino acid addition time; and (4) the reaction completion time. Measured variables for the dry and liquid lysine solutions are reported in Table 9.

TABLE 9
Characteristics of 3.0% B:Lys solution

| | Item | | Value |
|---|---|---|---|
| pH | | | 6.77 |
| Density (kg/L (lb/gal) | | | 1.2 (9.6) |
| | Viscosity (cps) | 30 rpm | 3.20 |
| | at −4.5° C. | 60 rpm | 3.80 |
| | (23.9° F.) | 80 rpm | 4.05 |
| | | 120 rpm | 5.10 |

| | |
|---|---|
| Element (wt. %) | 2.1 |
| Nitrogen (wt. %) | 3.9 |

TABLE 10
Time/temperature data

| Time (mm:ss) | Temperature (° C. (° F.)) | Process Notes |
|---|---|---|
| 00:00 | 20.0 (68.0) | Add SOLUBOR |
| 00:30 | 22.1 (71.7) | |
| 1:00 | 24.9 (76.9) | |
| 1:30 | 29.8 (85.7) | |
| 2:00 | 33.8 (92.9) | |
| 2:30 | 38.3 (100.9) | |
| 3:00 | 42.1 (107.8) | Increase temperature |
| | | setting to 6 |
| 3:30 | 46.8 (116.3) | |
| 4:00 | 50.4 (122.7) | Decrease temperature |
| | | setting to 3 |
| 4:30 | 53.4 (128.2) | |
| 5:00 | 55.9 (132.7) | |
| 5:30 | 57.7 (135.9) | Add lysine |
| 6:30 | 54.2 (129.5) | |
| 7:30 | 51.3 (124.3) | |
| 8:30 | 49.4 (121.0) | |
| 9:00 | 49.3 (120.8) | |
| 9:30 | 49.3 (120.7) | |
| 10:00 | 49.2 (120.6) | |
| 10:30 | 49.3 (120.7) | |
| 11:00 | 49.8 (121.7) | |
| 11:30 | 49.9 (121.9) | |
| 12:00 | 50.3 (122.5) | |
| 12:30 | 50.8 (123.4) | |
| 13:00 | 51.1 (124.0) | Reaction completion |

During mixing of the solution, the formulation with liquid lysine salted out. Accordingly, liquid lysine is not preferred for use in the manufacture of a 3% B:Lys complex. The sodium borate completely dissolved when the temperature of the solution reached 60° C. (140° F.). The dry lysine was completely dissolved and reacted with the boron solution between 60° C. and 71.1° C. (140° F. and 160° F.). Maintaining this temperature range in the solution shortened the reaction time from 26 minutes to 13 minutes. Thus, when the temperature of the boron/water solution was held at 60° C. (140° F.), the reaction between boron and lysine drove to completion very quickly.

Sodium borate is poorly soluble at room temperature. Sodium borate is generally freely soluble in boiling water. In this Example, the dissolution time of sodium borate was decreased from 11 minutes to 5.5 minutes by increasing the temperature of the solution to 57.7° C. (135.9° F.). For scale up purposes, the temperature of the water will generally be increased to 60° C. (140° F.) prior to addition of sodium borate.

### Example 4. Absorption Efficacy of Boron Compositions

This Example measured the absorption of boron into the leaf of a plant using various commercial boron sources as compared to boron glycinate and boron lysinate. In particular, this Example considered the following compositions: (1) boric acid (10.0%); (2) ethanolamine boron (5.0%); (3) boric acid (5.0%) complexed with methylene diurea and methylene urea; (4) boron glycinate (3.0%); and (5) boron lysinate (2.5%).

The solutions were mixed with clean, fresh water at two rates: 0.41 ounces of boron product to 68 ounces of water (rate 1) and 1.22 ounces of boron product to 68 ounces of water. Each solution was applied independently of one another at these rates, and the applications emulated commercial application conditions, with 30-inch spacings. The test solutions were applied to three different fields to allow for data replication. The corn was at growth stage V5 during the test. Each test solution was evenly applied using a portable foliar applicator (MILWAUKEE M18 sprayer with a four-gallon tank). Application time per row was recorded to allow for the calculation of an average application rate of the applicator. Any remaining application solution was weighed to allow for the exact calculation of applied solution per acre (converted below to ounces per acre). The solution application rates are reported below in Table 11 and boron application rates are reported below in Table 12. Applications were done in the heat of the day (96° F.), with minimal wind to preclude drift. Control strips for each product at both application rates were identified, and tissue samples from the control strips were collected to reflect an application rate of 0.00 ounces of product per 68 ounces of water.

TABLE 11
Solution application rates

| | Soln. Application Rate, ozs/acre | |
|---|---|---|
| Solution | Rate 1 | Rate 2 |
| Boric Acid and Various Derivatives | | |
| Boric acid | 14.87 | 50.95 |
| Boric acid + methylene diurea, | 16.98 | 49.94 |
| methylene urea | | |
| Boron Containing Compounds | | |
| Boron ethanolamine | 15.82 | 47.66 |
| Boron:Amino Acid Complexes | | |
| Boron glycinate | 14.84 | 44.36 |
| Boron lysinate | 14.62 | 50.19 |

TABLE 12
Application rates adjusted for solution boron concentration

| | Boron Application Rate, ozs/acre | |
|---|---|---|
| Item | Rate 1 | Rate 2 |
| Boric Acid and Various Derivatives | | |
| Boric acid | 1.70 | 5.04 |
| Boric acid + methylene diurea, | .85 | 2.49 |
| methylene urea | | |
| Boron ethanolamine | .79 | 2.38 |
| Boron:Amino Acid Complexes | | |
| Boron glycinate | .44 | 1.51 |
| Boron lysinate | .37 | 1.11 |

Tissues were collected after two days using standard tissue collection procedures. Twelve top collar leaves were collected per row. The leaf samples were placed in paper bags and sent to a commercial lab for leaf mineral analysis using ICP-AA.

The application rates reported above Table 11 were adjusted by the concentration of the applied product so that the data could be compared on an equivalent basis, which are reported in Table 12. Tissue boron was regressed against the adjusted applied rates using the Fit Y×X model of SAS JMP (v. 15.0). The Experimental Unit was Field.

Regression models are reported in Table 12. Five chemical compounds containing boron were evaluated. The slope of the line was defined according to the following equation:

$$Boron Absorption = \frac{Δ Tissue Boron}{ΔApplied Boron}$$

Table 13 reports the regression models for the various chemical compounds and formulations evaluation in the Example.

TABLE 13
Absorption efficacy of boron compounds

| | B Absorption, | | |
|---|---|---|---|
| Item | ppm/oz applied B | r² | p-value |
| Boric Acid and Various Derivatives | | | |
| Boric acid | 4.58 | .97 | <.0001 |
| Boric acid + methylene di-urea, | 3.81 | .68 | <.0001 |
| methylene urea | | | |
| Boron Containing Compounds | | | |
| Boron ethanolamine | 4.13 | .92 | <.0001 |
| Boron:Amino Acid Complexes | | | |
| Boron glycinate | 4.07 | .81 | <.0001 |
| Boron lysinate | 7.66 | .94 | <.0001 |

The use of boric acid (10%) resulted in an increase of 4.58 ppm of corn tissue boron for every ounce of boron applied. Attaching a methylene diurea and methylene urea molecule to the boric acid decreased the absorption of boron into the tissue by 0.77 ppm. Attaching an ethanolamine molecule to boric acid decreased the absorption of boron into the tissue by 0.45 ppm.

An insoluble crystal structure precipitated from the boron glycinate solution. Without being bound to a particular theory, it is believed that the insoluble crystal structure is boric acid. It is possible that some boric acid did complex with glycine; however, the boron glycinate did not absorb as well as boric acid (0.51 ppm/oz B less). Applying boron lysinate to the corn plant resulted in a boron absorption of 7.66 ppm of boron for every ounce of applied boron. This was 3.92 ppm greater than boric acid.

### Example 5. Absorption Efficacy of Boron Compositions

The same procedure of Example 4 was followed for this Example, and the same application solutions at rate 2 were used and applied to three separate fields of corn at growth stage V7. Each test solution was evenly applied using a portable foliar applicator (MILWAUKEE M18 sprayer with a four-gallon tank). The products were applied according to the rates reported in Table 13.

TABLE 14
Solution application rates

Application Rate, ozs/acre

| Item | Mixed Solution | Applied Boron |
|---|---|---|
| Boric acid | 7.96 | .796 |
| Boron Lysinate | 7.35 | .180 |

Tissues were collected after two days using standard tissue collection procedures. Twelve top collar leaves were collected per row. The leaf samples were placed in paper bags and sent to a commercial lab for leaf mineral analysis using ICP-AA.

Tissue boron data were analyzed using the Fit Y×X module of SAS JMP (v. 15.0). The tissue data were compared using the Oneway Analysis option. Means for boric acid and boron lysinate were separated using the Student's t-test. Results are reported in Table 15.

TABLE 15
Absorption of boric acid vs. boron lysinate

| Product | Boron Absorption, ppm/oz B | SEM |
|---|---|---|
| Boric acid | 4.97^{a} | 1.37 |
| Boron lysinate | 13.13^{b} | 2.68 |

^{a, b}Prob > | t | = .0285

The absorption of boric acid into the leaf tissue was similar to the results reported in Example 4 (4.58 vs. 4.97). Boron lysinate was more aggressively absorbed into the leaf in Example 5 compared to Example 4 (7.66 vs. 13.13). Without being bound to a particular theory, these data would suggest that the corn plant has a lysine requirement that increases as the plant matures. Similar to Example 4, the corn plant absorbed more boron per unit of applied boron with boron lysinate compared to the industry standard of boric acid. This result was significant (p<0.5). It is believed that the complex (i.e., chemical association) formed between boron and lysine is responsible for the increased uptake of boron using boron lysinate.

Based on the foregoing results, boron glycinate is less preferred from a commercial perspective than boron lysinate due to the formation of salt crystals as a result of the irreversible precipitation of boric acid from the solution. However, a portion of the glycine:boron complex may not precipitate and may still be remaining in solution and deliverable to the plant.

Boron is an essential micronutrient for production crops as it plays an essential role in the formation of cell walls and in the fruiting mechanism of plants. Boron is preferentially absorbed from the soil by the plants' root systems. The absorption rate of boron by the roots emulates the absorption rate of water. Once boron is absorbed by the root system, it is transported up the stem of the plant through the xylem. Once in the xylem, the free boron engages in its metabolic tasks.

In situations where boron is not freely available in the soil, it is common practice to apply boric acid to the leaf in an attempt to supplement the boron needs of the plant. Boric acid is drawn into the plant through passive permeation. Boric acid functions similar to urea in that it can be drawn into the plant due to a favorable permeability coefficient that allows the weak acid to permeate the lipid bilayer. There also may be some limited active transport of boric acid by aquaporins. Aquaporins are proteins that transport a broad range of solutes across the lipid bilayer, including water, urea, glycerol, polyols, purines, and pyrimidines. Interestingly, aquaporins do not transport amino acids.

Once inside the plant, boric acid complexes with various sugar alcohols to form esters of cis-diols. These cis-diols move the boric acid through the stem using the xylem as the transport system. The formation of these complexes has another biological advantage: the lowering of the pKₐ of the boric acid. The lowering of the pKₐ of boric acid allows for an easier dissociation of the element boron from the acid structure.

The supplementation of boron using boric acid is an inefficient process that relies on favorable passive permeability through the lipid bilayer of the leaf tissue and the formation of the borate-diol complexes to ease the passage of boron into the metabolism of the plant. There is some active transport of boric acid, but the boric acid competes with other molecules for this transport mechanism.

One problem with supplementing boron as boric acid through the leaf is that the boric acid does not move from the phloem into the xylem. In other words, the boric acid that absorbs in the leaf stays in the leaf. The reason for the immobility of the boric acid from leaf to stem is due to the concentration gradient of boron in the leaf versus boric acid in the stem. Boron that has been absorbed through the root as boric acid complexes with various sugars, which reduces the amount of freely available circulating boric acid. This creates a concentration gradient between the xylem and phloem that is energetically not advantageous to the plant to transfer the boron (as boric acid) from the leaf into the stem. As a result, the boric acid stays in the leaf, circulating with the phloem.

Experimental data such as those reported herein can be difficult to evaluate unless the data is normalized. In the present case, the application rates were normalized by the concentration of the product so that the data were analyzed by comparing the increase in leaf tissue boron against the amount of boron provided to the leaf. This normalization allows for a product with an 8% boron concentration to be compared to a product with 2.5% boron concentration.

Boric acid relies on passive transport through the lipid bilayer using pressure gradients. In Example 4, sufficient boric acid was absorbed into the leaf to raise the leaf tissue boron by 4.58 ppm per ounce applied boron. Attaching a urea or urea-containing molecule did not improve the boric acid absorption. Attaching a methylene diurea and methylene urea compound to the boric acid decreased the amount of boron absorbed into the leaf by 0.77 ppm.

While it is not known precisely how ethanolamine is absorbed into the plant, it is hypothesized that the general class of aquaporin transport proteins take ethanolamine into the plant through the lipid bilayer. In this test, attaching boron to ethanolamine decreased the leaf tissue boron by 0.45 ppm compared to boric acid. The reduction in boron absorption when boron is tied to ethanolamine suggests that the ethanolamine molecule does not participate in an active transport mechanism with an aquaporin.

Glycine is an amino acid and, in most cases, it can be synthesized in the plant from serine, another amino acid. Additionally, there is a transport protein that pulls glycine through the lipid bi-layer and directly into plant. A glycine:boron compound precipitates once the reaction has come to completion. However, it is hypothesized that some portion of the glycine:boron complex may still remain in the solution and will not precipitate. In this test, the solution that was tested decreased the amount of boron in the leaf by 0.51 ppm compared to boric acid. Without being bound to a particular theory, it is believed that, in at least this Example, boron did not form a complex with glycine.

The formation of a chemical bond between boron and lysine does not result in the formation of an insoluble precipitate. It is hypothesized that the reason why the reaction between lysine and boron dies not result in this precipitation is that the boron, as a form of boric acid, forms a complex with lysine due to the ionic bond formed with either the alpha- or zeta-amine groups.

In Example 4, the boron from boron lysinate increased tissue boron by 7.66 ppm. This was 3.08 ppm greater than boric acid. It is hypothesized that the increase in the amount of boron delivered to leaf tissue by boron lysinate occurred for two reasons: 1) the boron lysinate is neutral in charge, thereby negating the need to rely on a permeation coefficient; and 2) the Lyp1 transport protein actively transported the boron across the lipid bilayer using lysine.

To confirm the results from Example 4, Example 5 evaluated the two products that increased leaf tissue boron the most: boric acid and boron lysinate. In Example 5, boric acid was absorbed into the leaf at a similar rate compared to boric acid in Example 4. However, the boron lysinate was absorbed at a significantly higher rate compared to the boric acid (13.13 vs. 4.97) and against the absorption of the boron lysinate in Example 4 (13.13 vs. 7.66).

In Example 5, the corn was two growth stages more advanced compared to Example 4 (V5 vs. V7). For boric acid, this would suggest that the absorption rate is constant in corn, regardless of growth stage. This would support the theory that boric acid is absorbed into leaf tissue through passive permeability.

On the other hand, the absorption of boron lysinate into the leaf tissue was considerably greater at growth stage V7 compared to growth stage V5 (V7=13.13; V5=7.66). Without being bound to a particular theory, it is believed that the lysine nutritional requirement for corn increases as the plant matures, and as a result, the absorption rate of boron lysinate increases as well. It is well known in the field of animal nutrition that the lysine requirement of an animal changes with age and stage of production. This fundamental aspect of biology is not known in plant nutrition. These data would strongly support the concept that corn has a requirement for lysine, and that this requirement changes as the plant grows and matures.

The results from these two experiments demonstrate that the corn plant prefers a boron lysinate compared to all other commercially available boron product forms. This preference is driven by the presence of an active transport protein that actively pulls boron lysinate through the lipid bilayer and into the plant's metabolic functions. The corn plant increases the rate of absorption of boron lysinate as the corn plant grows and matures. This increase is driven by an increased nutritional requirement for both boron and lysine as the corn plant grows and matures.

### Example 6: Characterization of Boron Lysinate

Several studies were carried out to characterize an aqueous composition comprising boron lysinate, which was prepared as described in Example 3 above.

Prior to the characterization studies, small portion of the aqueous boron lysinate composition (referred to hereinafter as “Aqueous Boron Lysinate”) was removed and allowed to dry overnight in a fume hood while covered. Subsequent references of this material shall be referred to as “Dried Boron Lysinate.”

### FTIR Analysis

Dried Boron Lysinate was analyzed by FTIR, along with samples of lysine and borate for comparison.

The FTIR scans demonstrated a defined peak absorbance around a wavelength of 2900 cm⁻¹ which is consistent with the presence of lysine in both the Boron A++ and the Lysine HCl. Similar peaks were observed around the wavenumber of 1000 cm⁻¹ for both the Boron A++ and the Disodium Octaborate Tetrahydrate. This is consistent with the excess H⁺ present in the solution during the reaction. No evidence of covalent bond formation was measured using FT-IR, which supports the idea that meta-boric acid forms an ionic bond with either the alpha- or zeta-amino groups in lysine

The FTIR analysis therefore indicated that the Dried Boron Lysinate retained the similar or same organic bonds found in the parent lysine.

### TOF-MS Analysis

Dried Boron Lysinate (0.02549 g) was massed and dissolved in 25 mL of water. Separately, 20 μL of Aqueous Boron Lysinate was diluted in 20 mL of water. Lysine (0.02041 g) was dissolved in 20 mL of water. Sodium Borate (0.01996 g) was dissolved in 20 mL of water. Each solution was then analyzed by direct infusion on a time-of-flight mass spectrometer.

During TOF-MS direct infusion analysis, Aqueous Boron Lysinate produced significantly similar ions (−143 m/z and −145 m/z) as lysine (−145 m/z) and borate (−143 m/z). This suggests that the organic composition did not materially change between the two samples, and indicates that the boron lysinate is primarily an ionic association.

### NMR Analysis

Samples of Aqueous Boron Lysinate, Dried Boron Lysinate, tetrasodium octaborate, and lysine were subjected to nuclear magnetic resonance (NMR) analysis.

The ¹¹B NMR data showed similar spectra between Aqueous Boron Lysinate, Dried Boron Lysinate, and tetrasodium octaborate.

The ¹H NMR spectra were nearly identical between Aqueous Boron Lysinate and Lysine. As with the FTIR analysis, this suggests that the organic composition did not materially change between the two samples.

The ¹³C NMR data was also nearly identical between the two boron lysinate states and the lysine.

### Example 7: Application of Iron Lysinate and Magnesium Lysinate to Soybean

A composition comprising an about 50:50 molar blend of iron lysinate (1.2% w/w iron) and magnesium lysinate (1.5% w/w magnesium) was applied to a soybean field in Southwest Kansas. The blend was applied at a rate of approximately 3 gallons per acre over a three-week period. The field contained browned areas that were believed to be caused by iron chlorosis. Following application of the iron and magnesim lysinate, the browned areas experienced remarkable regrowth. Application of the lysinate blend mitigated the effect of chlorosis and significantly improved plant vigor 21 days after application.

### Example 8. Application of Iron Lysinate and Magnesium Lysinate to Sorghum

A composition comprising an about 50:50 blend of iron lysinate (1.2% w/w iron) and magnesium lysinate (1.5% w/w magnesium) was applied to a sorghum field in Central Kansas. The blend was applied at a rate of approximately 1 gallon per acre of a three-week period. The field contained browned areas that were believed to be caused by iron chlorosis. Following application of the iron and magnesium lysinate, the browned areas experienced remarkable regrowth. Application of the lysinate blend mitigated the effect of chlorosis and significantly improved plant vigor 21 days after application.

### Example 9: Application of Iron Lysinate and Magnesium Lysinate to Soybean

A composition comprising an about 50:50 blend of iron lysinate and magnesium lysinate was applied to a zone of a soybean field displaying chlorotic tissue. Results from this application as compared to non-treated non-chlorotic tissue and non-treated chlorotic tissue are provided in Table 11.

TABLE 16
Yield Results

| | Application | Average | Average | Total | | |
|---|---|---|---|---|---|---|
| | Rate | Moisture | Yield | Yield | Harvest | Area |
| Zone | (gal/acre) | (%) | (bu/ac) | (bu) | (ac) | (ac) |
| Non- | 0 | 10.92 | 71.13 | 4355 | 61.23 | 64.24 |
| chlorotic | | | | | | |
| Tissue | | | | | | |
| Chlorotic | 0 | 20.3 | 40.10 | 8 | 0.21 | 0.20 |
| Tissue | | | | | | |
| Treated | 2 | 14.62 | 68.14 | 322 | 4.73 | 4.71 |
| Chlorotic | | | | | | |
| Tissue | | | | | | |

Having described the invention in detail, it will be apparent that modifications and variations are possible without departing from the scope of the invention defined in the appended claims.

When introducing new elements of the present invention or the preferred embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements.

As various changes could be made in the above products without departing from the scope of the invention, it is intended that all matter contained in the above description and shown in the accompanying drawings shall be interpreted as illustrative and not in a limiting sense.

1. A mineral lysinate complex of Formula Ha: 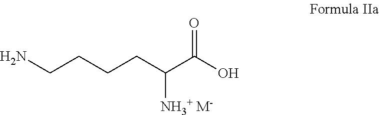
wherein M is a mineral or an anion comprising a mineral, wherein said mineral is selected from the group consisting of zinc, copper, manganese, boron, molybdenum, iron, potassium, or a combination thereof.

2. The mineral lysinate complex of claim 1 wherein M is boron or a boron-containing anion.

3. The mineral lysinate complex of claim 1 wherein the complex is a compound of Formula IIa-i:

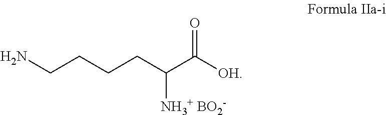

4. An aqueous agrochemical composition suitable for application to a plant, a seed, or soil, the composition comprising:
a mineral lysinate complex of claim 1 in a concentration of from about 1% to about 20% by weight of the composition; and
water, in a concentration of from about 80% to about 99% by weight of the composition.

5. The aqueous agrochemical composition of claim 4 wherein the composition is in the form of an aqueous solution.

6. The aqueous agrochemical composition of claim 4 further comprising at least one agriculturally acceptable surfactant.

7. A method of preparing a mineral amino acid complex, the method comprising:
an amino acid addition step wherein an amino acid is mixed with water, thereby forming a reaction mixture, and
a mineral reactant addition step wherein a mineral reactant is added to the reaction mixture,
wherein said amino acid is lysine;
and wherein said mineral reactant comprises boron.

8. The method of claim 7 wherein the amino acid addition step and the mineral reactant addition step are conducted simultaneously.

9. The method of claim 7 wherein the mineral reactant addition step is conducted after the amino acid addition step.

10. The method of claim 7 wherein the reaction mixture is heated during the mineral reactant addition step.

11. The method of claim 10 wherein the reaction mixture is heated to a temperature of between about 65° C. and about 75° C. during the mineral reactant addition step.

12. The method of claim 7 wherein the mineral reactant is a mineral salt comprising boron and a counterion.

13. The method of claim 7 wherein the mineral reactant is boric acid.

14. The method of claim 12 wherein the mineral salt comprises a mineral anion comprising boron, and a cationic counterion selected from the group consisting of hydrogen, an alkali metal, or an alkaline earth metal.

15. The method of claim 12 wherein the mineral salt is sodium borate.

16. The method of claim 12 wherein the mineral salt is a sodium borate mineral salt selected from the group consisting of sodium tetraborate decahydrate, disodium octaborate tetrahydrate, and mixtures thereof.

17. The method of claim 7 wherein the amino acid is added to the reaction mixture in an amount of at least about 5 percent by weight, relative to the total weight of the reaction mixture.

18. The method of claim 7 wherein the amino acid is added to the reaction mixture in an amount of from about 5 percent by weight to about 40 percent by weight, relative to the total weight of the reaction mixture.

19. The method of claim 7 wherein the mineral reactant is added to the reaction mixture in an amount of from about 10 percent by weight to about 30 percent by weight, relative to the total weight of the reaction mixture.

20. An aqueous agrochemical composition comprising:
a first mineral lysinate complex of Formula Ia wherein M is iron; 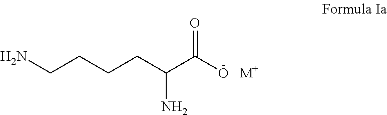
a second mineral lysinate complex of Formula Ia wherein M is manganese or magnesium;
and water.

21. The composition of claim 20 wherein in the second amino acid complex, M is magnesium.

22. The composition of claim 20 wherein the molar ratio of the first amino acid complex to the second amino acid complex is from about 2:1 to about 1:2.

23. The composition of claim 22 wherein the molar ratio is about 1:1.
